(19) United States
(12) Patent Application Publication
(10) Pub. No.: US 2026/0248700 A1
(43) Pub. Date: Aug. 27, 2026

(54) COMPOSITION FOR THE COSMETIC TREATMENT OF KERATIN FIBERS

(71) Applicant: BIOSYNTHIS, SAINT CYR SOUS DOURDAN (FR)

(72) Inventor: Thierry BERNOUD, SAINT CYR SOUS DOURDAN (FR)

(73) Assignee: BIOSYNTHIS, SAINT CYR SOUS DOURDAN (FR)

(21) Appl. No.: 18/854,686

(22) PCT Filed: Apr. 5, 2023
(86) PCT No.: PCT/EP2023/059073

§ 371 Date: Nov. 20, 2024

(30) Foreign Application Priority Data
Apr. 5, 2022 (FR) FR2203126

(51) Int. Cl. A61K 8/31; A61K 8/892; A61Q 5/12

(52) U.S. Cl. CPC A61K 8/31; A61K 8/892; A61Q 5/12; A61K 2800/30; A61K 2800/591

(57) ABSTRACT

A composition for the cosmetic treatment of keratin fibers includes: a fraction which includes at least one branched C₁₀ alkane having a flash point of less than or equal to 95° C. measured according to standard ASTM D93, a fraction which comprises at least one linear or branched, volatile C>10 alkane having a flash point of less than or equal to 95° C. measured according to standard ASTM D93, and at least one dimethiconol, and is free of volatile silicone oil.

This application concerns the technical field of cosmetics.

More specifically, the invention relates to a composition intended for the cosmetic treatment of keratin fibers, in particular hair.

The invention also relates to a method for the cosmetic treatment of keratin fibers in which a composition according to the invention is applied to said fibers, as well as the use of this composition for the care of keratin fibers, in particular hair.

As part of this request, a number of definitions must be given.

An “alkane” is a saturated hydrocarbon consisting only of carbon and hydrogen atoms linked together by simple covalent bonds whose general formula is CₙH₂ₙ₊₂.

A “straight alkane” is an alkane in which each carbon atom is bonded to a maximum of two carbon atoms.

A “branched alkane” is an alkane in which some carbon atoms are linked to three or even four carbon atoms.

A “Cₓ alkane” is an alkane consisting of x carbon atoms, and the alkane may be linear or branched. For example, n-dodecane is a C₁₂ alkane.

A “C>x alkane” is an alkane consisting of at least x+1 carbon atoms, and the alkane may be linear or branched. For example, n-dodecane is a C>10 linear alkane or C₁₂ alkane.

A “C≤x alkane” is an alkane consisting of at most x carbon atoms or less than x carbon atoms and the alkane may be linear or branched. For example, 2,3,6-trimethylheptane (CAS 4032-93-3) is a C≤10 alkane.

A “Cₓ-C_{y}” alkane is an alkane consisting of a number of carbon atoms between x and y inclusive. For example, C₁-C₂ alkanes are methane and ethane.

An “isoalkane” is a branched alkane containing at least one carbon atom linked to at least three carbon atoms including a methyl group.

Commonly, a “volatile linear alkane” is a linear alkane having a flash point less than or equal to 95° C. measured according to ASTM D93.

The flash point is the flash point measured according to ASTM D93. The unit of the flash point measured according to ASTM D93 is degrees Celsius (° C.). It can also be an average of values, each of the values being obtained by means of a measurement according to ASTM D93.

The term “mass percentage” refers to the ratio of the mass of a first compound to the total mass of a mixture of compounds or composition, reduced to a percentage. For example, if 10 grams of a compound y are present in a mixture z having a total mass of 100 grams, then the mass percentage of y in z is 10%.

A “bio-based” compound or organic composition is defined as one in which the organic carbon present in the compound or composition is of plant origin (1⁴C) by radiocarbon analysis according to one of the following standards: ASTM D6866, EN 16640 or EN 16785-1.

An organic compound or composition is qualified as bio-based if the organic carbon present in the compound or composition is 100% of plant origin, i.e. if the ratios ¹⁴C/¹²C or ¹⁴C/¹³ determined by radiocarbon analysis according to one of the following standards ASTM D6866, EN 16640 or EN 16785-1 correspond to the ratios of living plant products.

The “naturalness index” of an organic composition is the percentage of organic carbon of plant origin (¹⁴C) determined by radiocarbon analysis according to one of the following standards: ASTM D6866, EN 16640 or EN 16785-1.

For the purposes of the present invention, the OECD 301F method makes it possible to assess the biodegradability of a substance by a manometric respirometry test.

The use of volatile silicone oils in cosmetic products is set to disappear, in particular the use of cyclomethicones which are short-chain cyclic silicone compounds of formula [Si(CH₃)₂—O]ₙ with n between 3 and 7 (limits included). Indeed, their potential harmful effects on the environment, and even on human health, have been highlighted by several studies since 2005, the date of the first report on the toxicological risks to humans of cyclomethicones D4 and D5 by the Danish Environmental Protection Agency.

Research was then carried out in order to identify compounds likely to offer a satisfactory alternative, these compounds having in particular to have similar volatility characteristics and to allow the preparation of formulations having at least similar properties in terms of viscosity and sensory properties (ability to spread on hair and soft, “dry” and non-greasy feel of the film obtained).

For this purpose, various compositions including alkanes have been proposed.

Alkane-based compositions have been developed in the presence of carbonyl, carbonate or ester compounds.

For example, the use of a mixture of linear or branched dialkylcarbonate with an alkane, preferably branched and saturated, containing from 8 to 40 carbon atoms, was proposed in application US2005/0079986.

It has also been suggested in application US2004/0241200 to use a combination of a neopentyl glycol polyester, such as neopentyl glycol diheptanoate and an isoparaffin such as isododecane. This mixture is notably marketed by the company INOLEX under the trade name LexFeel® D4 and D5.

More recently, ester oils in association with one or more alkanes have been proposed.

Thus, patent application EP2623088A2 describes hair care compositions containing at least one ester oil and at least one isoparaffin chosen from the group consisting of isodecane, isoundecane, isotridecane and isotetradecane.

More recently, patent application DE102017223528A1 proposes to combine at least one alkane from the group consisting of undecane, dodecane and tridecane with at least one ester oil and one Crambe abyssinica seed oil.

Research has also highlighted the potential of alkane mixtures to reduce or potentially eliminate the use of volatile silicones in cosmetic products. Alkane mixtures of interest can be complex, include multiple compounds and multiple types of alkanes, both linear and branched.

For example, patent application WO2009/064790A1 uses a mixture of three alkane fractions: a C₁₂-C₁₄ isoalkane fraction, a C₁₃-C₁₅ isoalkane fraction and a so-called C₁₃-C₁₅ alkane mixture fraction in very specific proportions.

In the remainder of this application the term fraction used to define the different parts of a composition must be understood as a part of the composition, said parts or fractions being combined to define the composition in its entirety. The terms part and fraction may be used interchangeably.

Thus, the mixture described above comprises three parts or three fractions.

The Applicant in application WO2010/115973A1 proposed the use of a mixture of alkanes comprising at least one linear C₁₀-C₁₂ paraffin and at least one linear C₁₄-C₂₄ paraffin.

Application WO2019/197507A1 uses a mixture of alkanes comprising at least one C₈-C₁₀ alkane in combination with at least one C>11 alkane.

The multiple proposals made around the use of alkanes indicate that obtaining properties at least similar to those obtained using volatile silicone oils is a real challenge.

Recently, complex proposals have appeared for the preparation of compositions for hair treatment of interest. In particular, patent application DE102018220849A1 uses in association with at least one C₉-C₁₇ alkane, two types of fatty alcohols, at least one cationic surfactant of quaternary ammonium type and at least one monovalent C₂-C₈ alcohol.

More recently, it was even proposed in patent application FR3094639A1 to combine at least one branched C₈-C₁₆ alkane with three different types of non-volatile silicones in a hair composition.

There therefore remains a need to develop hair compositions exhibiting properties of interest in the absence of volatile silicone oils and this for various hair uses.

Surprisingly, a hair composition comprising at least one C₁₀-C₁₂ branched alkane, at least one volatile linear alkane and at least one dimethiconol exhibiting good cosmetic properties despite the absence of volatile silicone oils has been developed by the Applicant.

Their properties are comparable to those of compositions containing volatile silicone oils in terms of sensory properties, shine, softness, ease of detangling, protection during styling and smoothing without ever weighing down the hair.

The composition according to the invention makes it possible to condition keratin fibers in a very satisfactory manner. In particular, it makes it possible to significantly improve shine and softness. The keratin fibers remain light, supple, and non-greasy, neither visually nor to the touch.

Given the improvement in viscosity, it allows the formulation of compositions containing fewer non-volatile silicones and therefore improves the naturalness of the compositions.

,the ease of detangling hair, without weighing it down or making it greasy. The keratin fibers remain light, flexible, easy to style and shape. They are not greasy either visually or to the touch, and do not squeak to the touch.

Remarkably, the compositions for the cosmetic treatment of keratin fibers according to the invention can have a naturalness index greater than 85%.

The invention relates to a composition for the cosmetic treatment of keratin fibers characterized in that it comprises:
(a) a fraction A which comprises at least one branched C₁₀-C₁₂ alkane
b) at least one dimethiconol and is free of volatile silicone oil.

The invention particularly relates to a composition for the cosmetic treatment of keratin fibers characterized in that it comprises:
(a) a fraction A which comprises at least trimethylheptane,
(b) at least one dimethiconol, and is free of volatile silicone oil.

The invention particularly relates to a composition for the cosmetic treatment of keratin fibers characterized in that it comprises:
(a) a fraction A which comprises at least one branched C₁₀-C₁₂ alkane
(b) a fraction B which comprises at least one volatile linear or branched C>10 alkane
(c) at least one dimethiconol
   and is free of volatile silicone oil.

The invention relates to a composition for the cosmetic treatment of keratin fibers characterized in that it comprises:
a) a fraction A which comprises at least one branched C₁₀-C₁₂ alkane
a) a fraction B which comprises at least one volatile linear alkane
b) at least one dimethiconol
   and is free of volatile silicone oil.

The invention relates more particularly to a composition for the cosmetic treatment of keratin fibers characterized in that it comprises:
(a) a fraction A which comprises at least one branched C₁₀ alkane
(b) a fraction B which comprises at least one volatile linear or branched alkane C>10
(c) at least one dimethiconol
   and is free of volatile silicone oil.

In one embodiment, the composition for the cosmetic treatment of keratin fibers according to the invention is characterized in that it comprises a mass percentage of less than or equal to 15% of isododecane relative to the total mass of the composition.

In one embodiment, the composition for the cosmetic treatment of keratin fibers according to the invention is characterized in that it comprises a mass percentage of less than or equal to 10% of isododecane relative to the total mass of the composition.

In one embodiment, the composition for the cosmetic treatment of keratin fibers according to the invention is characterized in that it comprises a mass percentage of less than or equal to 10% of isododecane relative to the total mass of the composition.

Preferably, the composition for the cosmetic treatment of keratin fibers according to the invention is characterized in that it is free of isododecane.

The various components of the composition according to the invention are detailed below.

In one embodiment, fraction A and fraction B form a “volatile mixture of alkanes”.

In one embodiment, the cosmetic treatment composition for keratin fibers according to the invention is characterized in that the flash point of the mixture consisting of fraction A and fraction B is less than or equal to 50° C.

In a preferred embodiment, the cosmetic treatment composition for keratin fibers according to the invention is characterized in that the flash point of the mixture consisting of fraction A and fraction B is between 45 and 50° C.

In one embodiment, the cosmetic treatment composition for keratin fibers according to the invention is characterized in that the flash point of the mixture consisting of fraction A and fraction B is less than or equal to 45° C.

In one embodiment, the cosmetic treatment composition for keratin fibers according to the invention is characterized in that the flash point of the mixture consisting of fraction A and fraction B is between 4° and 45° C.

In one embodiment, the cosmetic treatment composition for keratin fibers according to the invention is characterized in that the flash point of the mixture consisting of fraction A and fraction B is less than or equal to 40° C.

In a preferred embodiment, the cosmetic treatment composition for keratin fibers according to the invention is characterized in that the flash point of the mixture consisting of fraction A and fraction B is between 35 and 40° C.

According to one embodiment, the cosmetic treatment composition for keratin fibers according to the invention is characterized in that the mixture consisting of fraction A and fraction B is free of alkanes whose number of carbon atoms is greater than 16.

According to one embodiment, the cosmetic treatment composition for keratin fibers according to the invention is characterized in that the mixture consisting of fraction A and fraction B is free of alkanes whose number of carbon atoms is greater than 15.

According to one embodiment, the cosmetic treatment composition for keratin fibers according to the invention is characterized in that the mixture consisting of fraction A and fraction B is free of alkanes whose number of carbon atoms is greater than 14.

In one embodiment, the cosmetic treatment composition for keratin fibers according to the invention is characterized in that the mixture consisting of fraction A and fraction B has a naturalness index greater than or equal to 60% carbon 14 (14C) determined according to one of the following standards: ASTM D6866, EN 16640 or EN 16785-1.

In one embodiment, the cosmetic treatment composition for keratin fibers according to the invention is characterized in that the mixture consisting of fraction A and fraction B has a naturalness index greater than or equal to 70% carbon 14 (14C) determined according to one of the following standards ASTM D6866, EN 16640 or EN 16785-1.

In one embodiment, the cosmetic treatment composition for keratin fibers according to the invention is characterized in that the mixture consisting of fraction A and fraction B has a naturalness index greater than or equal to 75% carbon 14 (14C) determined according to one of the following standards: ASTM D6866, EN 16640 or EN 16785-1.

In one embodiment, the cosmetic treatment composition for keratin fibers according to the invention is characterized in that the mixture consisting of fraction A and fraction B has a naturalness index greater than or equal to 85% carbon 14 (14C) determined according to one of the following standards: ASTM D6866, EN 16640 or EN 16785-1.

In one embodiment, the cosmetic treatment composition for keratin fibers according to the invention is characterized in that the mixture consisting of fraction A and fraction B has a naturalness index greater than or equal to 90% carbon 14 (14C) determined according to one of the following standards: ASTM D6866, EN 16640 or EN 16785-1.

In one embodiment, the cosmetic treatment composition for keratin fibers according to the invention is characterized in that the mixture consisting of fraction A and fraction B has a naturalness index of 100% carbon 14 (14C) determined according to one of the following standards: ASTM D6866, EN 16640 or EN 16785-1.

In one embodiment, the cosmetic treatment composition for keratin fibers according to the invention is characterized in that fraction A and fraction B are obtained from raw materials of plant, bacterial or animal origin.

In one embodiment, the cosmetic treatment composition for keratin fibers according to the invention is characterized in that fraction A and fraction B are obtained from raw materials of plant origin.

In one embodiment, the cosmetic treatment composition for keratin fibers according to the invention is characterized in that the mixture consisting of fraction A and fraction B has a biodegradability of at least 50% according to the OECD 301F method.

In one embodiment, the cosmetic treatment composition for keratin fibers according to the invention is characterized in that the mixture consisting of fraction A and fraction B has a biodegradability of at least 60% according to the OECD 301F method.

In one embodiment, the cosmetic treatment composition for keratin fibers according to the invention is characterized in that the mixture consisting of fraction A and fraction B has a biodegradability of between 50 and 80% according to the OECD 301F method.

In one embodiment, the cosmetic treatment composition for keratin fibers according to the invention is characterized in that the mixture consisting of fraction A and fraction B has a biodegradability of between 50 and 70% according to the OECD 301F method.

In one embodiment, the cosmetic treatment composition for keratin fibers according to the invention is characterized in that the mixture consisting of fraction A and fraction B has a biodegradability of between 50 and 60% according to the OECD 301F method.

In one embodiment, the cosmetic treatment composition for keratin fibers according to the invention is characterized in that the mixture consisting of fraction A and fraction B has a biodegradability of between 60 and 70% according to the OECD 301F method.

In one embodiment, the cosmetic treatment composition for keratin fibers according to the invention is characterized in that the mixture consisting of fraction A and fraction B has a biodegradability of between 70 and 80% according to the OECD 301F method.

In one embodiment, the cosmetic treatment composition for keratin fibers according to the invention is characterized in that the mass percentage of the mixture consisting of fraction A and fraction B is at least 10% relative to the total mass of the composition.

In one embodiment, the cosmetic treatment composition for keratin fibers according to the invention is characterized in that the mass percentage of the mixture consisting of fraction A and fraction B is at least 15% relative to the total mass of the composition.

In one embodiment, the cosmetic treatment composition for keratin fibers according to the invention is characterized in that the mass percentage of the mixture consisting of fraction A and fraction B is at least 20% relative to the total mass of the composition.

In one embodiment, the cosmetic treatment composition for keratin fibers according to the invention is characterized in that the mass percentage of the mixture consisting of fraction A and fraction B is at least 25% relative to the total mass of the composition.

In one embodiment, the cosmetic treatment composition for keratin fibers according to the invention is characterized in that the mass percentage of the mixture consisting of fraction A and fraction B is at least 50% relative to the total mass of the composition.

In one embodiment, the cosmetic treatment composition for keratin fibers according to the invention is characterized in that the mass percentage of the mixture consisting of fraction A and fraction B is between 25 and 80% relative to the total mass of the composition.

In one embodiment, the cosmetic treatment composition for keratin fibers according to the invention is characterized in that the mass percentage of the mixture consisting of fraction A and fraction B is between 40 and 80% relative to the total mass of the composition.

In one embodiment, the cosmetic treatment composition for keratin fibers according to the invention is characterized in that the mass percentage of the mixture consisting of fraction A and fraction B is between 25 and 60% relative to the total mass of the composition.

In one embodiment, the cosmetic treatment composition for keratin fibers according to the invention is characterized in that the mass percentage of the mixture consisting of fraction A and fraction B is between 30 and 50% relative to the total mass of the composition.

In one embodiment, the cosmetic treatment composition for keratin fibers according to the invention is characterized in that the mass percentage of the mixture consisting of fraction A and fraction B is between 40 and 60% relative to the total mass of the composition.

In one embodiment, the cosmetic treatment composition for keratin fibers according to the invention is characterized in that the mass percentage of the mixture consisting of fraction A and fraction B is between 60 and 80% relative to the total mass of the composition.

Fraction A of the composition for the cosmetic treatment of keratin fibers according to the invention is chosen from the group comprising branched C₁₀ alkanes, branched C₁₁ alkanes, branched C₁₂ alkanes, and mixtures thereof.

Fraction A of the composition for the cosmetic treatment of keratin fibers according to the invention comprises at least one branched C₁₀-C₁₂ alkane.

In one embodiment, the composition for the cosmetic treatment of keratin fibers according to the invention comprises at least one branched C₁₀-C₁₂ alkane chosen from the group of branched C₁₀ alkanes.

In one embodiment, the composition for the cosmetic treatment of keratin fibers according to the invention comprises at least one C₁₀ branched alkane chosen from the group consisting of: 2-methylnonane (CAS 871-83-0), 4-methylnonane (CAS 17301-94-9), 3-methylnonane (CAS 5911-04-6), 3-ethyloctane (CAS 5881-17-4), 2,2-dimethyloctane (CAS 15869-87-1), 2,3-dimethyloctane (CAS 7146-60-3), 2,5-dimethyloctane (CAS 15869-89-3), 3,5-dimethyloctane (CAS 15869-93-9), 4-propylheptane (CAS 3178-29-8), 3-ethyl-2-methylheptane (CAS 14676-29-0), 2,3,6-trimethylheptane (CAS 4032-93-3), 3,3,4-trimethylheptane (CAS 20278-87-9), 2,3,4-trimethylheptane (CAS 52896-95-4), 2,2,4-trimethylheptane (CAS: 14720-74-2), 3,3-diethylhexane (CAS 17302-02-2), 2,2,3,3-tetramethylhexane (CAS 13475-81-5), 3-ethyl-2,2,3-trimethylpentane (CAS 52897-17-3), 2,2,3-trimethylheptane (CAS 52896-92-1), 2,3,5-trimethylheptane (CAS 20278-85-7), 3,3-diethylhexane (CAS 17302-02-2), 2,2,3,3-tetramethylhexane (CAS 13475-81-5), 3-ethyl-2,2,3-trimethylpentane (CAS 52897-17-3), 3,3,5-trimethylheptane (CAS 7154-80-5), 2,5,5-trimethylheptane (CAS 1189-99-7), 2,4,6-trimethylheptane (CAS 2613-61-8), 3,4,5-trimethylheptane (CAS 20278-89-1), 2,2,6-trimethylheptane (CAS 1190-83-6), 2,4,5-trimethylheptane (CAS 20278-84-6), 2,4,4-trimethylheptane (CAS 4032-92-2), 2,3,3-trimethylheptane (CAS 52896-93-2), 3,4,4-trimethylheptane (CAS 20278-88-0), and mixtures thereof.

For the purposes of the present invention, the term “trimethylheptanes” means all C₇ alkanes branched by three methyl groups.

In one embodiment, the composition for the cosmetic treatment of keratin fibers according to the invention comprises at least one branched C₁₀-C₁₂ alkane chosen from the group consisting of trimethylheptanes.

In one embodiment, the composition for the cosmetic treatment of keratin fibers according to the invention comprises at least one C₁₀-C₁₂ branched alkane trimethylheptane chosen from the group of trimethylheptanes consisting of 2,2,3-trimethylheptane (CAS 52896-92-1), 2,3,5-trimethylheptane (CAS 20278-85-7), 2,3,6-trimethylheptane (CAS 4032-93-3), 3,3,4-trimethylheptane (CAS 20278-87-9), 2,3,4-trimethylheptane (CAS 52896-95-4), 2,2,4-trimethylheptane (CAS: 14720-74-2,3,3,5-trimethylheptane (CAS 7154-80-5), 2,5,5-trimethylheptane (CAS 1189-99-7), 2,4,6-trimethylheptane (CAS 2613-61-8), 3,4,5-trimethylheptane (CAS 20278-89-1), 2,2,6-trimethylheptane (CAS 1190-83-6), 2,4,5-trimethylheptane (CAS 20278-84-6), 2,4,4-trimethylheptane (CAS 4032-92-2), 2,3,3-trimethylheptane (CAS 52896-93-2), 3,4,4-trimethylheptane (CAS 20278-88-0), and mixtures thereof.

In one embodiment, the at least one C₁₀-C₁₂ branched alkane present in the composition according to the invention is a trimethylheptane.

In one embodiment, the at least one C₁₀-C₁₂ branched alkane present in the composition according to the invention is 2,3,6-trimethylheptane (CAS 4032-93-3).

In one embodiment, the at least one C₁₀-C₁₂ branched alkane present in the composition according to the invention is 2,3,5-trimethylheptane (CAS 20278-85-7).

In one embodiment, the at least one C₁₀-C₁₂ branched alkane present in the composition according to the invention is 2,2,3-trimethylheptane (CAS 52896-92-1).

In one embodiment, the at least one C₁₀-C₁₂ branched alkane present in the composition according to the invention is 3,3,4-trimethylheptane (CAS 20278-87-9).

In one embodiment, the at least one C₁₀-C₁₂ branched alkane present in the composition according to the invention is 2,3,4-trimethylheptane (CAS 52896-95-4).

In one embodiment, the at least one C₁₀-C₁₂ branched alkane present in the composition according to the invention is 2,2,4-trimethylheptane (CAS: 14720-74-2)

In one embodiment, the at least one C₁₀-C₁₂ branched alkane present in the composition according to the invention is 2,2,3-trimethylheptane (CAS 52896-92-1).

In one embodiment, the at least one C₁₀-C₁₂ branched alkane present in the composition according to the invention is 3,3,5-trimethylheptane (CAS 7154-80-5).

In one embodiment, the at least one C₁₀-C₁₂ branched alkane present in the composition according to the invention is 2,5,5-trimethylheptane (CAS 1189-99-7).

In one embodiment, the at least one C₁₀-C₁₂ branched alkane present in the composition according to the invention is 2,4,6-trimethylheptane (CAS 2613-61-8).

In one embodiment, the at least one C₁₀-C₁₂ branched alkane present in the composition according to the invention is 3,4,5-trimethylheptane (CAS 20278-89-1).

In one embodiment, the at least one C₁₀-C₁₂ branched alkane present in the composition according to the invention is 2,2,6-trimethylheptane (CAS 1190-83-6).

In one embodiment, the at least one C₁₀-C₁₂ branched alkane present in the composition according to the invention is 2,4,5-trimethylheptane (CAS 20278-84-6).

In one embodiment, the at least one C₁₀-C₁₂ branched alkane present in the composition according to the invention is 2,4,4-trimethylheptane (CAS 4032-92-2).

In one embodiment, the at least one C₁₀-C₁₂ branched alkane present in the composition according to the invention is 2,3,3-trimethylheptane (CAS 52896-93-2).

In one embodiment, the composition for the cosmetic treatment of keratin fibers according to the invention comprises at least one branched C₁₀-C₁₂ alkane chosen from the group of branched C₁₁ alkanes.

In one embodiment, the composition for the cosmetic treatment of keratin fibers according to the invention comprises at least one branched C₁₀-C₁₂ alkane chosen from the group of branched C₁₂ alkanes.

In one embodiment, the composition for the cosmetic treatment of keratin fibers according to the invention comprises at least one C₁₀-C₁₂ branched alkane chosen from the group of C₁₂ branched alkanes consisting of: 2-methylundecane (CAS 31807-55-3), 3-methylundecane (CAS 1002-43-3), 4-methylundecane (CAS 2980-69-0), 5-methylundecane (CAS 1632-70-8), 6-methylundecane (CAS 17302-33-9), 2,4-dimethyldecane (CAS 2801-84-5), 4,4-dimethyldecane (CAS 17312-39-9), 3,5-dimethyldecane (CAS 17312-48-0), 2,5-dimethyldecane (CAS 17312-50-4), 2,3-dimethyldecane (17312-44-6), 3,3-dimethyldecane (17302-38-4), 3,7-dimethyldecane (CAS 17312-54-8), 3,4,6-trimethylnonane (CAS 62184-24-1), 3,5,6-trimethylnonane (CAS 62184-26-3), 3,5,7-trimethylnonane (CAS 62184-27-4), 2,5,7-trimethylnonane (CAS 62184-14-9), 2,5,6-trimethylnonane (CAS 62184-13-8), 2,5,7-trimethylnonane (CAS 62184-14-9), 2,5,8-trimethylnonane (CAS 49557-09-7), 3,3,4,5-tetramethyloctane (CAS 62185-21-1), 2,3,4,5-tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetramethyloctane (CAS 35866-96-7), 3-ethyl-4-methylnonane (CAS 62184-45-6), 3-ethyl-4,5-dimethyloctane (CAS 62183-72-6), 2,5-dimethyl-6-ethyloctane (CAS 62183-50-0), 2,2,4,6,6-pentamethylheptane (CAS 31807-55-3) or isododecane, and mixtures thereof.

In one embodiment, the composition for the cosmetic treatment of keratin fibers according to the invention does not comprise isododecane.

In one embodiment, the composition for the cosmetic treatment of keratin fibers according to the invention is characterized in that the at least one C₁₀-C₁₂ branched alkane has a naturalness index greater than or equal to 80% carbon 14 (1⁴C) determined according to one of the following standards ASTM D6866, EN 16640 or EN 16785-1.

Advantageously, 100% of the at least one C₁₀-C₁₂ branched alkane present in the composition for the cosmetic treatment of keratin fibers according to the invention has a carbon 14 (1⁴C) naturalness index determined according to one of the following standards: ASTM D6866, EN 16640 or EN 16785-1.

In one embodiment, the composition for the cosmetic treatment of keratin fibers according to the invention is characterized in that the mass percentage of fraction A is at least 20% relative to the mass of the mixture consisting of fraction A and fraction B.

In one embodiment, the composition for the cosmetic treatment of keratin fibers according to the invention is characterized in that the mass percentage of fraction A is greater than 50% relative to the mass of the mixture consisting of fraction A and fraction B.

In one embodiment, the composition for the cosmetic treatment of keratin fibers according to the invention is characterized in that the mass percentage of fraction A is between 20 and 100% relative to the mass of the mixture consisting of fraction A and fraction B.

In one embodiment, the composition for the cosmetic treatment of keratin fibers according to the invention is characterized in that the mass percentage of fraction A is between 20 and 70% relative to the mass of the mixture consisting of fraction A and fraction B.

In one embodiment, the composition for the cosmetic treatment of keratin fibers according to the invention is characterized in that the mass percentage of fraction A is between 51 and 100% relative to the mass of the mixture consisting of fraction A and fraction B.

In one embodiment, the composition for the cosmetic treatment of keratin fibers according to the invention is characterized in that the mass percentage of fraction A is between 30 and 60% relative to the mass of the mixture consisting of fraction A and fraction B.

In one embodiment, the composition for the cosmetic treatment of keratin fibers according to the invention is characterized in that the mass percentage of fraction A is between 40 and 70% relative to the mass of the mixture consisting of fraction A and fraction B.

In one embodiment, the composition for the cosmetic treatment of keratin fibers according to the invention is characterized in that the mass percentage of fraction A is between 70 and 100% relative to the mass of the mixture consisting of fraction A and fraction B.

In one embodiment, the composition for the cosmetic treatment of keratin fibers according to the invention is characterized in that the mass percentage of fraction A is between 30 and 50% relative to the mass of the mixture consisting of fraction A and fraction B.

In one embodiment, the composition for the cosmetic treatment of keratin fibers according to the invention is characterized in that the mass percentage of fraction A is between 40 and 60% relative to the mass of the mixture consisting of fraction A and fraction B.

In one embodiment, the composition for the cosmetic treatment of keratin fibers according to the invention is characterized in that the mass percentage of fraction A is between 50 and 70% relative to the mass of the mixture consisting of fraction A and fraction B.

In one embodiment, the composition for the cosmetic treatment of keratin fibers according to the invention is characterized in that the mass percentage of fraction A is between 80 and 100% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is at least 20% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is greater than 50% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 20% and 100% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 20% and 70% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 51% and 100% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 30% and 60% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 40% and 70% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 70% and 100% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 30% and 50% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 40% and 60% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 50% and 70% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 70% and 90% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is between 80% and 100% relative to the mass of the mixture consisting of fraction A and fraction B.

In one embodiment, the lipstick composition according to the invention is characterized in that the at least one branched C<10 alkane of fraction A of said volatile mixture of alkanes is 2,3,6-trimethylheptane (CAS 4032-93-3).

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is at least 20% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 20% and 100% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 20% and 70% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 51% and 100% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 30% and 60% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 40% and 70% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 70% and 100% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 30% and 50% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 40% and 60% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 50% and 70% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 70% and 90% relative to the mass of the mixture consisting of fraction A and fraction B.

In one embodiment, the composition for the cosmetic treatment of keratin fibers according to the invention is characterized in that the mass percentage of 2,3,6-trimethylheptane (CAS 4032-93-3) of the at least one branched C₁₀-C₁₂ alkane is between 80% and 100% relative to the mass of the mixture consisting of fraction A and fraction B.

In one embodiment, the composition for the cosmetic treatment of keratin fibers according to the invention is characterized in that the mass percentage of the at least one branched C₁₀-C₁₂ alkane is at least 10% relative to the total mass of the composition.

In one embodiment, the composition for the cosmetic treatment of keratin fibers according to the invention is characterized in that the mass percentage of the at least one branched C₁₀-C₁₂ alkane is greater than 20% relative to the total mass of the composition.

In one embodiment, the composition for the cosmetic treatment of keratin fibers according to the invention is characterized in that the mass percentage of the at least one branched C₁₀-C₁₂ alkane is between 20 and 60% relative to the total mass of the composition.

In one embodiment, the composition for the cosmetic treatment of keratin fibers according to the invention is characterized in that the mass percentage of the at least one branched C₁₀-C₁₂ alkane is between 20 and 50% relative to the total mass of the composition.

In one embodiment, the composition for the cosmetic treatment of keratin fibers according to the invention is characterized in that the mass percentage of the at least one branched C₁₀-C₁₂ alkane is between 30 and 50% relative to the total mass of the composition.

In one embodiment, the composition for the cosmetic treatment of keratin fibers according to the invention is characterized in that the mass percentage of the at least one branched C₁₀-C₁₂ alkane is between 20 and 30% relative to the total mass of the composition.

In one embodiment, the composition for the cosmetic treatment of keratin fibers according to the invention is characterized in that the mass percentage of the at least one branched C₁₀-C₁₂ alkane is between 30 and 40% relative to the total mass of the composition.

In one embodiment, the composition for the cosmetic treatment of keratin fibers according to the invention is characterized in that the mass percentage of the at least one branched C₁₀-C₁₂ alkane is between 40 and 50% relative to the total mass of the composition.

In one embodiment, the composition for the cosmetic treatment of keratin fibers according to the invention is characterized in that the mass percentage of the at least one branched C₁₀-C₁₂ alkane is between 50 and 60% relative to the total mass of the composition.

Fraction B of the composition for the cosmetic treatment of keratin fibers according to the invention comprises at least one volatile linear alkane.

A volatile linear alkane is defined as a linear alkane having a flash point less than or equal to 95° C. according to the ASTM D93 standard.

In one embodiment, the composition for the cosmetic treatment of keratin fibers according to the invention comprises at least one volatile linear alkane having a flash point less than or equal to 55° C. according to the ASTM D93 standard.

In one embodiment, the composition for the cosmetic treatment of keratin fibers according to the invention is characterized in that the at least one volatile linear alkane is chosen from the group comprising C₇, C₈, C₉, C₁₀, C₁₁, C₁₂, C₁₃ alkanes and their mixtures.

In one embodiment, the composition for the cosmetic treatment of keratin fibers according to the invention is characterized in that the at least one volatile linear alkane is chosen from the group consisting of n-heptane (CAS 142-82-5), n-octane (CAS 111-65-9), n-nonane (CAS 111-84-2), n-decane (CAS 124-18-5), n-undecane (CAS 1120-21-4), n-dodecane (CAS 112-40-3), n-tridecane (CAS 629-50-4) and mixtures thereof.

In one embodiment, the composition for the cosmetic treatment of keratin fibers according to the invention is characterized in that the at least one volatile linear alkane is n-decane (CAS 124-18-5).

In one embodiment, the composition for the cosmetic treatment of keratin fibers according to the invention is characterized in that the at least one volatile linear alkane is n-undecane (CAS 1120-21-4).

In one embodiment, the composition for the cosmetic treatment of keratin fibers according to the invention is characterized in that the at least one volatile linear alkane is n-dodecane (CAS 112-40-3).

In one embodiment, the composition for the cosmetic treatment of keratin fibers according to the invention is characterized in that the at least one volatile linear alkane is n-tridecane (CAS 629-50-4).

In one embodiment, fraction B of the composition for the cosmetic treatment of keratin fibers according to the invention comprises at least one volatile branched alkane having a flash point less than or equal to 95° C. according to the ASTM D93 standard.

In one embodiment, the composition for the cosmetic treatment of keratin fibers according to the invention is characterized in that the at least one branched alkane having a flash point less than or equal to 95° C. according to the ASTM D93 standard is chosen from the group comprising branched C₁₂ alkanes, branched C₁₃ alkanes, branched C₁₄ alkanes, branched C₁₅ alkanes, branched C₁₆ alkanes, and mixtures thereof.

In one embodiment, the composition for the cosmetic treatment of keratin fibers according to the invention is characterized in that the at least one branched alkane having a flash point less than or equal to 95° C. according to the ASTM D93 standard is a branched C₁₁ alkane.

In one embodiment, the composition for the cosmetic treatment of keratin fibers according to the invention is characterized in that the at least one branched alkane having a flash point less than or equal to 95° C. according to the ASTM D93 standard is a branched C₁₂ alkane.

In one embodiment, the composition for the cosmetic treatment of keratin fibers according to the invention is characterized in that the at least one branched alkane having a flash point less than or equal to 95° C. according to the ASTM D93 standard is a branched C₁₂ alkane chosen from the group consisting of: 3-methylundecane (CAS 1002-43-3), 4-methylundecane (CAS 2980-69-0), 5-methylundecane (CAS 1632-70-8), 6-methylundecane (CAS 17302-33-9), 2,4-dimethyldecane (CAS 2801-84-5), 4,4-dimethyldecane (CAS 17312-39-9), 3,5-dimethyldecane (17312-48-0), 2,5-dimethyldecane (CAS 17312-50-4), 2,3-dimethyldecane (17312-44-6), 3,3-dimethyldecane (17302-38-4), 3,7-dimethyldecane (CAS 17312-54-8), 3,4,6-trimethylnonane (CAS 62184-24-1), 3,5,6-trimethylnonane (CAS 62184-26-3), 3,5,7-trimethylnonane (CAS 62184-27-4), 2,5,7-trimethylnonane (CAS 62184-14-9), 2,5,6-trimethylnonane (CAS 62184-13-8), 2,5,7-trimethylnonane (CAS 62184-14-9), 2,5,8-trimethylnonane (CAS 49557-09-7), 3,3,4,5-tetramethyloctane (CAS 62185-21-1), 2,3,4,5-tetramethyloctane (CAS 62199-27-3), 2,2,4,5-tetramethyloctane (CAS 62183-80-6), 2,2,5,7-tetramethyloctane (CAS 62199-19-3), 2,3,4,7-tetramethyloctane (CAS 62199-29-5), 2,4,4,7-tetramethyloctane (CAS 35866-96-7), 3-ethyl-4-methylnonane (CAS 62184-45-6), 3-ethyl-4,5-dimethyloctane (CAS 62183-72-6), 2,5-dimethyl-6-ethyloctane (CAS 62183-50-0), 2,2,4,6,6-pentamethylheptane (CAS 13475-82-6) and mixtures thereof.

In one embodiment, the composition for the cosmetic treatment of keratin fibers according to the invention is characterized in that the at least one branched alkane having a flash point less than or equal to 95° C. according to the ASTM D93 standard is at least one isododecane.

In one embodiment, the composition for the cosmetic treatment of keratin fibers according to the invention is characterized in that the at least one branched alkane having a flash point less than or equal to 95° C. according to the ASTM D93 standard is 2,2,4,6,6-pentamethylheptane (CAS 13475-82-6).

In one embodiment, the composition for the cosmetic treatment of keratin fibers according to the invention is characterized in that the at least one branched alkane having a flash point less than or equal to 95° C. according to the ASTM D93 standard is a branched C₁₃ alkane.

In one embodiment, the composition for the cosmetic treatment of keratin fibers according to the invention is characterized in that the at least one branched alkane having a flash point less than or equal to 95° C. according to the ASTM D93 standard is a branched C₁₄ alkane.

In one embodiment, the composition for the cosmetic treatment of keratin fibers according to the invention is characterized in that the mass percentage of fraction B is between 0 and 80% relative to the mass of the mixture consisting of fraction A and fraction B.

In one embodiment, the composition for the cosmetic treatment of keratin fibers according to the invention is characterized in that the mass percentage of fraction B is between 0 and 50% relative to the mass of the mixture consisting of fraction A and fraction B.

In one embodiment, the composition for the cosmetic treatment of keratin fibers according to the invention is characterized in that the mass percentage of fraction B is between 0 and 30% relative to the mass of the mixture consisting of fraction A and fraction B.

In one embodiment, the composition for the cosmetic treatment of keratin fibers according to the invention is characterized in that the mass percentage of fraction B is at least 30% relative to the mass of the mixture consisting of fraction A and fraction B.

In one embodiment, the composition for the cosmetic treatment of keratin fibers according to the invention is characterized in that the mass percentage of fraction B is between 30 and 80% relative to the mass of the mixture consisting of fraction A and fraction B.

In one embodiment, the composition for the cosmetic treatment of keratin fibers according to the invention is characterized in that the mass percentage of fraction B is between 30 and 60% relative to the mass of the mixture consisting of fraction A and fraction B.

In one embodiment, the composition for the cosmetic treatment of keratin fibers according to the invention is characterized in that the mass percentage of fraction B is between 50 and 80% relative to the mass of the mixture consisting of fraction A and fraction B.

In one embodiment, the composition for the cosmetic treatment of keratin fibers according to the invention is characterized in that the mass percentage of fraction B is between 30 and 50% relative to the mass of the mixture consisting of fraction A and fraction B.

In one embodiment, the composition for the cosmetic treatment of keratin fibers according to the invention is characterized in that the mass percentage of fraction B is between 40 and 60% relative to the mass of the mixture consisting of fraction A and fraction B.

In one embodiment, the composition for the cosmetic treatment of keratin fibers according to the invention is characterized in that the mass percentage of fraction B is between 50 and 70% relative to the mass of the mixture consisting of fraction A and fraction B.

In one embodiment, the composition for the cosmetic treatment of keratin fibers according to the invention is characterized in that the mass percentage of fraction B is between 60 and 80% relative to the mass of the mixture consisting of fraction A and fraction B.

In one embodiment, the composition for the cosmetic treatment of keratin fibers according to the invention is characterized in that the mass percentage of fraction B is between 0 and 10% relative to the mass of the mixture consisting of fraction A and fraction B.

In one embodiment, the composition for the cosmetic treatment of keratin fibers according to the invention is characterized in that the mass percentage of fraction B is between 10 and 20% relative to the mass of the mixture consisting of fraction A and fraction B.

In one embodiment, the composition for the cosmetic treatment of keratin fibers according to the invention is characterized in that the mass percentage of fraction B is between 40 and 50% relative to the mass of the mixture consisting of fraction A and fraction B.

In one embodiment, the composition for the cosmetic treatment of keratin fibers according to the invention is characterized in that the mass percentage of fraction B is between 60 and 70% relative to the mass of the mixture consisting of fraction A and fraction B.

In one embodiment, the composition for the cosmetic treatment of keratin fibers according to the invention is characterized in that the mass percentage of fraction B is between 70 and 80% relative to the mass of the mixture consisting of fraction A and fraction B.

In one embodiment, the composition for the cosmetic treatment of keratin fibers according to the invention is characterized in that the mass percentage of the at least one volatile linear alkane is between 0 and 80% relative to the mass of the mixture consisting of fraction A and fraction B.

In one embodiment, the composition for the cosmetic treatment of keratin fibers according to the invention is characterized in that the mass percentage of the at least one volatile linear alkane is between 0 and 50% relative to the mass of the mixture consisting of fraction A and fraction B.

In one embodiment, the composition for the cosmetic treatment of keratin fibers according to the invention is characterized in that the mass percentage of the at least one volatile linear alkane is between 0 and 30% relative to the mass of the mixture consisting of fraction A and fraction B.

In one embodiment, the composition for the cosmetic treatment of keratin fibers according to the invention is characterized in that the mass percentage of the at least one volatile linear alkane is at least 30% relative to the mass of the mixture consisting of fraction A and fraction B.

In one embodiment, the composition for the cosmetic treatment of keratin fibers according to the invention is characterized in that the mass percentage of the at least one volatile linear alkane is between 30 and 80% relative to the mass of the mixture consisting of fraction A and fraction B.

In one embodiment, the composition for the cosmetic treatment of keratin fibers according to the invention is characterized in that the mass percentage of the at least one volatile linear alkane is between 30 and 60% relative to the mass of the mixture consisting of fraction A and fraction B.

In one embodiment, the composition for the cosmetic treatment of keratin fibers according to the invention is characterized in that the mass percentage of the at least one volatile linear alkane is between 50 and 80% relative to the mass of the mixture consisting of fraction A and fraction B.

In one embodiment, the composition for the cosmetic treatment of keratin fibers according to the invention is characterized in that the mass percentage of the at least one volatile linear alkane is between 30 and 50% relative to the mass of the mixture consisting of fraction A and fraction B.

In one embodiment, the composition for the cosmetic treatment of keratin fibers according to the invention is characterized in that the mass percentage of the at least one volatile linear alkane is between 40 and 60% relative to the mass of the mixture consisting of fraction A and fraction B.

In one embodiment, the composition for the cosmetic treatment of keratin fibers according to the invention is characterized in that the mass percentage of the at least one volatile linear alkane is between 50 and 70% relative to the mass of the mixture consisting of fraction A and fraction B.

In one embodiment, the composition for the cosmetic treatment of keratin fibers according to the invention is characterized in that the mass percentage of the at least one volatile linear alkane is between 60 and 80% relative to the mass of the mixture consisting of fraction A and fraction B.

In one embodiment, the composition for the cosmetic treatment of keratin fibers according to the invention is characterized in that the mass percentage of the at least one volatile linear alkane is between 0 and 10% relative to the mass of the mixture consisting of fraction A and fraction B.

In one embodiment, the composition for the cosmetic treatment of keratin fibers according to the invention is characterized in that the mass percentage of the at least one volatile linear alkane is between 10 and 20% relative to the mass of the mixture consisting of fraction A and fraction B.

In one embodiment, the composition for the cosmetic treatment of keratin fibers according to the invention is characterized in that the mass percentage of the at least one volatile linear alkane is between 40 and 50% relative to the mass of the mixture consisting of fraction A and fraction B.

In one embodiment, the composition for the cosmetic treatment of keratin fibers according to the invention is characterized in that the mass percentage of the at least one volatile linear alkane is between 60 and 70% relative to the mass of the mixture consisting of fraction A and fraction B.

In one embodiment, the composition for the cosmetic treatment of keratin fibers according to the invention is characterized in that the mass percentage of the at least one volatile linear alkane is between 70 and 80% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 0 and 30% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is at least 10% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is at least 30% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 10 and 80% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 30 and 80% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 30 and 60% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 50 and 80% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 30 and 50% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 40 and 60% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 50 and 70% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 60 and 80% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 0 and 10% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 10 and 20% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 20 and 30% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 40 and 50% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 50 and 60% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 60 and 70% relative to the mass of the mixture consisting of fraction A and fraction B.

In one embodiment, the composition for the cosmetic treatment of keratin fibers according to the invention is characterized in that the mass percentage of n-dodecane (CAS 112-40-3) of the at least one volatile linear alkane is between 70 and 80% relative to the mass of the mixture consisting of fraction A and fraction B.

In one embodiment, the composition for the cosmetic treatment of keratin fibers according to the invention is characterized in that the at least one volatile linear alkane has a naturalness index greater than or equal to 80% carbon 14 (¹⁴C) determined according to one of the following standards ASTM D6866, EN 16640 or EN 16785-1.

Advantageously, the composition for the cosmetic treatment of keratin fibers according to the invention is characterized in that the at least one volatile linear alkane has a naturalness index of 100% carbon 14 (¹⁴C) determined according to one of the following standards ASTM D6866, EN 16640 or EN 16785-1.

In one embodiment, the composition for the cosmetic treatment of keratin fibers according to the invention is characterized in that the mass percentage of the at least one volatile linear alkane is at least 10% relative to the total mass of the composition.

In one embodiment, the composition for the cosmetic treatment of keratin fibers according to the invention is characterized in that the mass percentage of the at least one volatile linear alkane is greater than or equal to 20% relative to the total mass of the composition.

In one embodiment, the composition for the cosmetic treatment of keratin fibers according to the invention is characterized in that the mass percentage of the at least one volatile linear alkane is between 20 and 60% relative to the total mass of the composition.

In one embodiment, the composition for the cosmetic treatment of keratin fibers according to the invention is characterized in that the mass percentage of the at least one volatile linear alkane is between 30 and 50% relative to the total mass of the composition.

In one embodiment, the composition for the cosmetic treatment of keratin fibers according to the invention is characterized in that the mass percentage of the at least one volatile linear alkane is between 20 and 30% relative to the total mass of the composition.

In one embodiment, the composition for the cosmetic treatment of keratin fibers according to the invention is characterized in that the mass percentage of the at least one volatile linear alkane is between 30 and 40% relative to the total mass of the composition.

In one embodiment, the composition for the cosmetic treatment of keratin fibers according to the invention is characterized in that the mass percentage of the at least one volatile linear alkane is between 40 and 50% relative to the total mass of the composition.

In one embodiment, the composition for the cosmetic treatment of keratin fibers according to the invention is characterized in that the mass percentage of the at least one volatile linear alkane is between 50 and 60% relative to the total mass of the composition.

The composition for the cosmetic treatment of keratin fibers according to the invention is characterized in that it comprises at least one dimethiconol.

Dimethiconol is a polydimethylsiloxane or PDMS polymer with dimethylsilanol chain ends.

In one embodiment, the dimethiconol has a molar mass by weight (Mw) greater than 1,000 g/mol.

In one embodiment, the dimethiconol has a molar mass by weight (Mw) greater than 50,000 g/mol.

In one embodiment, the dimethiconol has a molar mass by weight (Mw) greater than 100,000 g/mol.

In one embodiment, the composition for the cosmetic treatment of keratin fibers according to the invention is characterized in that it comprises at least one dimethiconol chosen from the group consisting of dimethiconols sold by the companies BRB International and DOW CORNING.

In one embodiment, the composition for the cosmetic treatment of keratin fibers according to the invention is characterized in that the mass percentage of the at least one dimethiconol is at least 2% relative to the total mass of the composition.

In one embodiment, the composition for the cosmetic treatment of keratin fibers according to the invention is characterized in that the mass percentage of at least one dimethiconol is greater than 4% relative to the total mass of the composition.

In one embodiment, the composition for the cosmetic treatment of keratin fibers according to the invention is characterized in that the mass percentage of the at least one dimethiconol is between 2 and 15% relative to the total mass of the composition.

In one embodiment, the composition for the cosmetic treatment of keratin fibers according to the invention is characterized in that the mass percentage of the at least one dimethiconol is between 2 and 20% relative to the total mass of the composition.

In one embodiment, the composition for the cosmetic treatment of keratin fibers according to the invention is characterized in that the mass percentage of the at least one dimethiconol is between 3 and 12% relative to the total mass of the composition.

In one embodiment, the composition for the cosmetic treatment of keratin fibers according to the invention is characterized in that the mass percentage of the at least one dimethiconol is between 4 and 9% relative to the total mass of the composition.

In one embodiment, the composition for the cosmetic treatment of keratin fibers according to the invention is characterized in that it comprises an alkane chosen from the group consisting of squalane, squalene and cyclododecane.

In one embodiment, the composition for the cosmetic treatment of keratin fibers according to the invention is characterized in that it comprises squalane.

In one embodiment, the composition for the cosmetic treatment of keratin fibers according to the invention is characterized in that it comprises squalene.

In one embodiment, the composition for the cosmetic treatment of keratin fibers according to the invention is characterized in that it comprises cyclododecane.

In one embodiment, the composition for the cosmetic treatment of keratin fibers according to the invention is characterized in that it comprises squalane and cyclododecane.

In one embodiment, the composition for the cosmetic treatment of keratin fibers according to the invention is characterized in that it comprises squalene and cyclododecane.

In one embodiment, the composition for the cosmetic treatment of keratin fibers according to the invention is characterized in that the mass percentage of squalane is between 2 and 15% relative to the total mass of the composition.

In one embodiment, the composition for the cosmetic treatment of keratin fibers according to the invention is characterized in that the mass percentage of squalane is between 3 and 12% relative to the total mass of the composition.

In one embodiment, the composition for the cosmetic treatment of keratin fibers according to the invention is characterized in that the mass percentage of squalane is between 4 and 9% relative to the total mass of the composition.

In one embodiment, the composition for the cosmetic treatment of keratin fibers according to the invention is characterized in that the mass percentage of squalene is between 2 and 15% relative to the total mass of the composition.

In one embodiment, the composition for the cosmetic treatment of keratin fibers according to the invention is characterized in that the mass percentage of squalene is between 3 and 12% relative to the total mass of the composition.

In one embodiment, the composition for the cosmetic treatment of keratin fibers according to the invention is characterized in that the mass percentage of squalene is between 4 and 9% relative to the total mass of the composition.

In one embodiment, the composition for the cosmetic treatment of keratin fibers according to the invention is characterized in that the mass percentage of cyclododecane is between 2 and 15% relative to the total mass of the composition.

In one embodiment, the composition for the cosmetic treatment of keratin fibers according to the invention is characterized in that the mass percentage of cyclododecane is between 3 and 12% relative to the total mass of the composition.

In one embodiment, the composition for the cosmetic treatment of keratin fibers according to the invention is characterized in that the mass percentage of cyclododecane is between 4 and 9% relative to the total mass of the composition.

In one embodiment, the composition for the cosmetic treatment of keratin fibers according to the invention is characterized in that it comprises at least one branched C₁₀-C₁₂ alkane, a volatile linear alkane, a dimethiconol and squalane.

In one embodiment, the composition for the cosmetic treatment of keratin fibers according to the invention is characterized in that it comprises at least one branched C₁₀-C₁₂ alkane, a volatile linear alkane, a dimethiconol and squalene.

In one embodiment, the composition for the cosmetic treatment of keratin fibers according to the invention is characterized in that it comprises at least one branched C₁₀-C₁₂ alkane, a volatile linear alkane, a dimethiconol and cyclododecane.

In one embodiment, the composition for the cosmetic treatment of keratin fibers according to the invention is characterized in that it comprises at least one branched C₁₀-C₁₂ alkane, a volatile linear alkane, a dimethiconol, cyclododecane and squalane.

In one embodiment, the composition for the cosmetic treatment of keratin fibers according to the invention is characterized in that it comprises at least one branched C₁₀-C₁₂ alkane, a volatile linear alkane, a dimethiconol, at least cyclododecane and squalene.

In one embodiment, the composition for the cosmetic treatment of keratin fibers according to the invention is characterized in that it comprises at least one trimethylheptane, one volatile linear alkane and one dimethiconol.

In one embodiment, the composition for the cosmetic treatment of keratin fibers according to the invention is characterized in that it comprises at least one trimethylheptane, one volatile linear alkane, one dimethiconol and cyclododecane.

In one embodiment, the composition for the cosmetic treatment of keratin fibers according to the invention is characterized in that it comprises at least one trimethylheptane, one volatile linear alkane, one dimethiconol and squalane.

In one embodiment, the composition for the cosmetic treatment of keratin fibers according to the invention is characterized in that it comprises at least one trimethylheptane, one volatile linear alkane, one dimethiconol and squalene.

In one embodiment, the composition for the cosmetic treatment of keratin fibers according to the invention is characterized in that it comprises at least one trimethylheptane, one volatile linear alkane, one dimethiconol, squalane and cyclododecane.

In one embodiment, the composition for the cosmetic treatment of keratin fibers according to the invention is characterized in that it comprises at least one trimethylheptane, one volatile linear alkane, one dimethiconol, squalene and cyclododecane.

In one embodiment, the composition for the cosmetic treatment of keratin fibers according to the invention is characterized in that it comprises at least one trimethylheptane, one volatile branched alkane and one dimethiconol.

In one embodiment, the composition for the cosmetic treatment of keratin fibers according to the invention is characterized in that it comprises at least one trimethylheptane, one volatile branched alkane, one dimethiconol and cyclododecane.

In one embodiment, the composition for the cosmetic treatment of keratin fibers according to the invention is characterized in that it comprises at least one trimethylheptane, one volatile branched alkane, one dimethiconol and squalane.

In one embodiment, the composition for the cosmetic treatment of keratin fibers according to the invention is characterized in that it comprises at least one trimethylheptane, one volatile branched alkane, one dimethiconol and squalene.

In one embodiment, the composition for the cosmetic treatment of keratin fibers according to the invention is characterized in that it comprises at least one trimethylheptane, one volatile branched alkane, one dimethiconol, squalane and cyclododecane.

In one embodiment, the composition for the cosmetic treatment of keratin fibers according to the invention is characterized in that it comprises at least one trimethylheptane, one volatile branched alkane, one dimethiconol, squalene and cyclododecane.

In one embodiment, the composition for the cosmetic treatment of keratin fibers according to the invention is characterized in that it comprises at least one trimethylheptane, n-dodecane and one dimethiconol.

In one embodiment, the composition for the cosmetic treatment of keratin fibers according to the invention is characterized in that it comprises at least one trimethylheptane, n-dodecane, one dimethiconol and cyclododecane.

In one embodiment, the composition for the cosmetic treatment of keratin fibers according to the invention is characterized in that it comprises at least one trimethylheptane, n-dodecane, one dimethiconol and squalane.

In one embodiment, the composition for the cosmetic treatment of keratin fibers according to the invention is characterized in that it comprises at least one trimethylheptane, n-dodecane, one dimethiconol and squalene.

In one embodiment, the composition for the cosmetic treatment of keratin fibers according to the invention is characterized in that it comprises at least one trimethylheptane, n-dodecane, one dimethiconol, squalane and cyclododecane.

In one embodiment, the composition for the cosmetic treatment of keratin fibers according to the invention is characterized in that it comprises at least one trimethylheptane, n-dodecane, one dimethiconol, squalene and cyclododecane.

In one embodiment, the composition for the cosmetic treatment of keratin fibers according to the invention is characterized in that it comprises at least 2,3,6-trimethylheptane, n-dodecane and a dimethiconol.

In one embodiment, the composition for the cosmetic treatment of keratin fibers according to the invention is characterized in that it comprises at least 2,3,6-trimethylheptane, n-dodecane, a dimethiconol and cyclododecane.

In one embodiment, the composition for the cosmetic treatment of keratin fibers according to the invention is characterized in that it comprises at least 2,3,6-trimethylheptane, n-dodecane, a dimethiconol and squalane.

In one embodiment, the composition for the cosmetic treatment of keratin fibers according to the invention is characterized in that it comprises at least 2,3,6-trimethylheptane, n-dodecane, a dimethiconol and squalene.

In one embodiment, the composition for the cosmetic treatment of keratin fibers according to the invention is characterized in that it comprises at least 2,3,6-trimethylheptane, n-dodecane, a dimethiconol, squalane and cyclododecane.

In one embodiment, the composition for the cosmetic treatment of keratin fibers according to the invention is characterized in that it comprises at least 2,3,6-trimethylheptane, n-dodecane, a dimethiconol, squalene and cyclododecane.

In one embodiment, the composition for the cosmetic treatment of keratin fibers according to the invention is characterized in that it comprises:
30% to 50% of 2,3,6-trimethylheptane (CAS 4032-93-3)
30% to 50% n-dodecane (CAS 112-40-3)
4% to 10% dimethiconol.

In one embodiment, the composition for the cosmetic treatment of keratin fibers according to the invention is characterized in that it comprises:
30% to 50% of 2,3,6-trimethylheptane (CAS 4032-93-3)
30% to 50% n-dodecane (CAS 112-40-3)
4% to 10% dimethiconol
5% to 15% dimethicone.

In one embodiment, the composition for the cosmetic treatment of keratin fibers according to the invention is characterized in that it comprises:
30% to 50% of 2,3,6-trimethylheptane (CAS 4032-93-3)
30% to 50% n-dodecane (CAS 112-40-3)
5% to 10% dimethiconol
5% to 10% cyclododecane

In one embodiment, the composition for the cosmetic treatment of keratin fibers according to the invention is characterized in that it comprises:
30% to 40% of 2,3,6-trimethylheptane (CAS 4032-93-3)
30% to 40% n-dodecane (CAS 112-40-3)
5% to 10% dimethiconol
5% to 10% cyclododecane
5% to 10% squalene

In one embodiment, the composition for the cosmetic treatment of keratin fibers according to the invention is characterized in that it comprises:
30% to 40% of 2,3,6-trimethylheptane (CAS 4032-93-3)
30% to 40% n-dodecane (CAS 112-40-3)
5% to 10% dimethiconol
5% to 10% cyclododecane
5% to 10% squalane

In one embodiment, the composition for the cosmetic treatment of keratin fibers according to the invention is characterized in that at least 50% by mass of its ingredients is of natural origin relative to the total mass of the composition.

In one embodiment, the composition for the cosmetic treatment of keratin fibers according to the invention is characterized in that at least 60% by mass of its ingredients is of natural origin relative to the total mass of the composition.

In one embodiment, the composition for the cosmetic treatment of keratin fibers according to the invention is characterized in that at least 70% by mass of its ingredients is of natural origin relative to the total mass of the composition.

In one embodiment, the composition for the cosmetic treatment of keratin fibers according to the invention is characterized in that at least 80% by mass of its ingredients is of natural origin relative to the total mass of the composition.

In one embodiment, the composition for the cosmetic treatment of keratin fibers according to the invention is characterized in that at least 90% by mass of its ingredients is of natural origin relative to the total mass of the composition.

In one embodiment, the composition for treating keratin fibers according to the invention comprises at least one non-volatile oil.

For the purposes of the present invention, the term “non-volatile oil” means an oil having a flash point greater than 110° C. measured according to the ATSM D93 standard.

These oils can be of vegetable, mineral or synthetic origin.

In one embodiment, the composition for treating keratin fibers according to the invention comprises at least one non-volatile oil chosen from the group of natural oils.

In one embodiment, the composition for treating keratin fibers according to the invention comprises at least one non-volatile oil chosen from the group of natural oils of plant origin.

In one embodiment, the composition for treating keratin fibers according to the invention comprises at least one natural non-volatile oil of plant origin selected from the group consisting of camellia oil, sweet almond oil, avocado oil, castor oil, olive oil, jojoba oil, sunflower oil, sesame oil, soybean oil, wheat germ oil, rice bran oil, corn germ oil, passionflower oil, rye oil, peanut oil, grape seed oil, corn oil, rapeseed oil, safflower oil, coconut oil, hazelnut oil, palm oil, shea butter oil, amaranth seed oil, beauty leaf oil, blackberry seed oil, linseed oil, macadamia nut oil, corn germ oil, marula oil, evening primrose oil, brazil nut oil, pecan nut oil, copra oil, peach kernel oil, sea buckthorn pulp oil, grape seed oil, walnut oil, wild rose oil, rosehip oil, argan oil, camelina oil, pracaxi oil, ximenia oil, lavender oil, rosemary oil, geranium oil, evening primrose oil, thistle oil, calophyllum oil, chamomile oil, cedarwood oil, clary sage oil, sandalwood oil, kernel oil apricot, amaranth seed oil, babassu oil, cottonseed oil, borage seed oil, pomegranate seed oil, grapefruit seed oil, hemp seed oil and elderberry seed oil.

In one embodiment, the composition for treating keratin fibers according to the invention comprises at least one non-volatile oil of mineral origin.

In one embodiment, the composition for treating keratin fibers according to the invention comprises at least one non-volatile oil of mineral origin chosen from the group consisting of paraffin oil and vaseline oil.

In a preferred embodiment, the composition for treating keratin fibers according to the invention comprises at least one natural non-volatile oil.

In one embodiment, the composition for treating keratin fibers according to the invention comprises at least one non-volatile oil chosen from the group of synthetic oils.

In one embodiment, the composition for treating keratin fibers according to the invention comprises at least one synthetic non-volatile oil chosen from the group consisting of transesterified vegetable oils, fluorinated oils and fatty esters.

In one embodiment, the composition for treating keratin fibers according to the invention comprises at least one non-volatile oil chosen from the group of synthetic oils consisting of synthetic triglyceride oils.

In one embodiment, the composition for treating keratin fibers according to the invention comprises at least one non-volatile synthetic triglyceride oil selected from the group consisting of capric/caprylic triglycerides with unbranched fatty acid residues and glyceryl triisostearin with branched fatty acid residues.

In one embodiment, the composition for treating keratin fibers according to the invention comprises at least one synthetic non-volatile oil based on hydrocarbons.

In one embodiment, the synthetic non-volatile oil based on hydrocarbons is selected from the group of monoesters of saturated or unsaturated fatty carboxylic acids, comprising from 2 to 30 carbon atoms, which can carry an alcohol function, with linear, branched, saturated or unsaturated fatty alcohols carrying from 2 to 30 carbon atoms.

In one embodiment, the synthetic non-volatile hydrocarbon-based oil is selected from the group of monoesters of saturated or unsaturated fatty carboxylic acids, comprising at most 8 carbon atoms, with linear, branched, saturated or unsaturated fatty alcohols bearing from 2 to 30 carbon atoms.

In one embodiment, the synthetic monoester-based non-volatile oil is selected from the group consisting of cetyl 2-ethylhexanoate, 2-hexyldecylstearate, 2-hexyldecyllaurate, isodecyl neopentanoate, isononyl isononanoate, 2-ethylhexyl palmitate, 2-ethylhexyl stearate, isopropyl myristate, isopropyl palmitate, isopropyl stearate, isopropyl isostearate, isopropyl oleate, butyl stearate, isooctyl stearate, octyl stearate (purcellin oil), isononyl stearate, isocetyl stearate, isononyl isononanoate, isotridecyl isononanoate, cetearyl isononanoate, hexyl laurate, 2-ethylhexyl laurate, 2-ethylhexyl isostearate, 2-octyldecyl myristate, isostearyl neopentanoate, tridecyl neopentanoate, 2-ethylhexyl cocoate, 2-octyldodecyl palmitate, butyloctanoic acid-2-butyloctanoate, diisotridecyl acetate, n-butyl stearate, n-hexyl laurate, n-decyl oleate, oleyl oleate, oleyl erucate, erucyl oleate and erucyl erucate.

In one embodiment, the synthetic monoester-based non-volatile oil is selected from the group of benzoic acid alkyl esters.

In one embodiment, the synthetic monoester-based non-volatile oil is selected from the group of C₁₂-C₁₅ benzoic acid alkyl esters.

In one embodiment, the C₁₂C₁₅ alkyl ester of benzoic acid is selected from the group consisting of isostearyl ester of benzoic acid, ethylhexyl benzoate and octyldodecyl ester of benzoic acid.

In one embodiment, the composition for treating keratin fibers according to the invention comprises at least one synthetic non-volatile oil chosen from the group of dicarboxylic acid esters.

In one embodiment, the composition for treating keratin fibers according to the invention comprises at least one non-volatile dicarboxylic acid ester oil selected from the group consisting of di-n-butyl adipate, di-(2-ethylhexyl) adipate, di-isopropyl adipate, dioctyl adipate, di-(2-ethylhexyl) succinate, diisooctyl succinate, di-(2-hexyldecyl) succinate, di-isotridecyl acetate, diethyl sebacate, di-n-butyl sebacate, dioctyl sebacate, dioctyl malate, dioctyl maleate and dicaprylyl maleate.

In one embodiment, the composition for treating keratin fibers according to the invention comprises at least one synthetic non-volatile oil chosen from the group of diol esters.

In one embodiment, the composition for treating keratin fibers according to the invention comprises at least one non-volatile oil of diol esters chosen from the group consisting of ethylene glycol dioleate, ethylene glycol diisotridecanoate, ethylene glycol dipalmitate, propylene glycol di-(2-ethylhexanoate), propylene glycol diisostearate, propylene glycol di-pelargonate, butanediol diisostearate and neopentyl glycol dicaprylate.

In one embodiment, the composition for treating keratin fibers according to the invention comprises at least one synthetic non-volatile oil chosen from the group consisting of symmetrical, asymmetrical or cyclic esters of carbonic acid with fatty alcohols.

In one embodiment, the composition for treating keratin fibers according to the invention comprises at least one synthetic non-volatile oil chosen from the group consisting of symmetrical, asymmetrical or cyclic esters of carbonic acid with C₃-C₂₂ monoalcohols, diols or triols.

In one embodiment, the composition for treating keratin fibers according to the invention comprises at least one synthetic non-volatile oil chosen from the group consisting of glycerol carbonate and dicaprylyl carbonate.

In one embodiment, the composition for treating keratin fibers according to the invention comprises at least one synthetic non-volatile oil selected from the group of non-volatile polyorganosiloxanes other than a dimethiconol. Non-volatile polyorganosiloxane oil is understood to mean a silicone-based oil incapable of evaporating on contact with the skin or hair in less than one hour.

In one embodiment, the non-volatile silicone-based oil has a saturation vapor pressure of less than 0.13 Pa (0.01 mm Hg).

In one embodiment, the non-volatile silicone-based oil has a weight average molar mass (Mw) of at least 50,000 g/mol.

In one embodiment, the non-volatile silicone-based oil has a weight average molar mass (Mw) of between 50,000 and 1,500,000 g/mol.

In one embodiment, the non-volatile silicone-based oil has a weight average molar mass (Mw) of between 200,000 and 1,000,000 g/mol.

In one embodiment, the non-volatile silicone-based oil is selected from polydiaIkylsiloxanes.

In one embodiment, the non-volatile silicone-based oil is at least one polydialkylsiloxane selected from compounds known as dimethicone and PDMS.

In one embodiment, the non-volatile silicone-based oil is chosen from polydimethylsiloxanes carrying trimethylsilyl groups at the end of the chain.

In one embodiment, the non-volatile silicone-based oil is liquid at 25° C. and atmospheric pressure (1.013×10⁵ Pa).

In one embodiment, the composition for treating keratin fibers according to the invention is characterized in that the mass proportion of the non-volatile silicone-based oil is between 2 and 20% relative to the total mass of the composition.

In one embodiment, the composition for treating keratin fibers according to the invention is characterized in that the mass proportion of the non-volatile silicone-based oil is between 4 and 15% relative to the total mass of the composition.

In one embodiment, the composition for treating keratin fibers according to the invention is characterized in that the mass proportion of the non-volatile silicone-based oil is between 5 and 13% relative to the total mass of the composition.

In one embodiment, the composition for treating keratin fibers according to the invention comprises at least one natural non-volatile oil and at least one synthetic non-volatile oil.

In one embodiment, the composition for treating keratin fibers according to the invention comprises at least one natural non-volatile oil and does not comprise a synthetic non-volatile oil.

In one embodiment, the composition for treating keratin fibers according to the invention comprises at least one synthetic non-volatile oil and does not comprise any natural non-volatile oil.

In a preferred embodiment, the composition for treating keratin fibers according to the invention comprises at least one natural non-volatile oil.

In one embodiment, the composition for treating keratin fibers according to the invention is characterized in that the mass proportion of the at least one non-volatile oil is between 0.5 and 20% relative to the total mass of the composition.

In one embodiment, the composition for treating keratin fibers according to the invention is characterized in that the mass proportion of the at least one non-volatile oil is between 2 and 15% relative to the total mass of the composition.

In one embodiment, the composition for treating keratin fibers according to the invention is characterized in that the mass proportion of the at least one non-volatile oil is between 3 and 10% relative to the total mass of the composition.

In one embodiment, the composition for treating keratin fibers according to the invention comprises at least one wax.

“Wax” is a lipophilic compound that has a reversible solid/liquid state change. In other words, the compound is solid at room temperature (at 25° C.) and has a melting point greater than or equal to 30° C.

In one embodiment, the composition for treating keratin fibers according to the invention comprises at least one wax chosen from the group consisting of esters of linear or branched saturated or unsaturated C₂₂-C₄₀ fatty alcohol with a linear or branched saturated or unsaturated C₁₂-C₂₀ fatty acid.

In one embodiment, the composition for treating keratin fibers according to the invention comprises at least one wax chosen from the group consisting of esters of linear saturated C₂₈-C₃₂ fatty alcohol with a linear saturated C₁₄-C₁₈ fatty acid.

In one embodiment, the composition for treating keratin fibers according to the invention comprises at least one wax chosen from the group consisting of linear or branched, saturated or unsaturated C₂₂-C₃₄ fatty acids.

In one embodiment, the composition for treating keratin fibers according to the invention comprises two different waxes selected from the group consisting of esters of linear or branched saturated or unsaturated C₂₂-C₄₀ fatty alcohol with a linear or branched saturated or unsaturated C₁₂-C₂₀ fatty acid and linear or branched, saturated or unsaturated C₂₂-C₃₄ fatty acids.

In one embodiment, the composition for treating keratin fibers according to the invention comprises at least one wax chosen from the group consisting of carnauba waxes, esparto waxes, Japanese waxes, cork waxes, sugar cane waxes, Ouricury waxes, Montan waxes, sunflower waxes and raffia wax.

In one embodiment, the composition for treating keratin fibers according to the invention comprises at least one wax chosen from fruit waxes.

In one embodiment, the composition for treating keratin fibers according to the invention comprises at least one wax chosen from animal waxes.

In one embodiment, the composition for treating keratin fibers according to the invention comprises at least one animal wax chosen from the group consisting of insect waxes such as beeswax and shellac wax.

In one embodiment, the composition for treating keratin fibers according to the invention comprises at least one animal wax chosen from the group consisting of spermaceti, wool wax, lanolin wax and lanolin derivatives.

In one embodiment, the composition for treating keratin fibers according to the invention comprises at least one mineral wax.

In one embodiment, the composition for treating keratin fibers according to the invention comprises at least one mineral wax chosen from the group consisting of paraffin wax, vaseline wax, lignite wax, microcrystalline waxes, ceresin and ozokerite.

In one embodiment, the composition for treating keratin fibers according to the invention comprises at least one wax chosen from hydrogenated or hardened waxes.

In one embodiment, the composition for treating keratin fibers according to the invention comprises at least one wax chosen from chemically modified waxes.

In one embodiment, the composition for treating keratin fibers according to the invention comprises at least one chemically modified wax selected from the group consisting of montane ester waxes, Sasol waxes and hydrogenated jojoba waxes.

In one embodiment, the composition for treating keratin fibers according to the invention comprises at least one wax chosen from the group consisting of triglycerides of saturated and optionally hydroxylated C₁₆-C₃₀ fatty acids.

In one embodiment, the composition for treating keratin fibers according to the invention comprises at least one wax selected from the group consisting of hardened triglyceride fats such as hydrogenated palm oil, hydrogenated coconut oil and hydrogenated castor oil.

In one embodiment, the composition for treating keratin fibers according to the invention comprises at least one wax selected from the group consisting of glyceryl tribehenate, glyceryl tri-12-hydroxystearate, complete synthetic esters of fatty acids and glycols or C₂-C₆ polyols, monoalkanolamides of fatty acids comprising a C₁₂-C₂₂ acyl radical and a C₂-C₄ alkanol radical, esters of aromatic carboxylic acids, dicarboxylic acids or hydroxycarboxylic acids (for example 12-hydroxystearic acid) and saturated and/or unsaturated, branched and/or unbranched C₁₀-C₈₀ alcohols, lactides of long-chain hydroxycarboxylic acids and complete esters of alcohols fatty and di- and tricarboxylic acids.

In one embodiment, the composition for treating keratin fibers according to the invention comprises at least one wax chosen from vegetable waxes.

In one embodiment, the composition for treating keratin fibers according to the invention comprises at least one vegetable wax chosen from the group consisting of carnauba wax, sunflower wax, rice wax, apple wax, Candellila wax, Ouricury wax, Japanese wax, cocoa butter, cork fiber waxes and sugar cane waxes.

In one embodiment, the composition for treating keratin fibers according to the invention comprises at least one synthetic wax.

In one embodiment, the composition for treating keratin fibers according to the invention comprises at least one synthetic wax chosen from the group consisting of polyethylene waxes and Fischer-Tropsch waxes.

In one embodiment, the composition for treating keratin fibers according to the invention comprises at least one surfactant or emulsifier.

In one embodiment, the composition for treating keratin fibers according to the invention comprises at least one surfactant chosen from neutral or nonionic, cationic, anionic and zwitterionic or amphoteric surfactants.

The surfactant can be of plant, animal or synthetic origin.

In one embodiment, the composition for treating keratin fibers according to the invention comprises at least one surfactant of natural origin.

In one embodiment, the composition for treating keratin fibers according to the invention comprises at least one surfactant of plant origin.

In one embodiment, the composition for treating keratin fibers according to the invention comprises at least one surfactant of animal origin.

In one embodiment, the composition for treating keratin fibers according to the invention comprises at least one surfactant of synthetic origin.

In one embodiment, the composition for treating keratin fibers according to the invention comprises at least one cationic surfactant.

In one embodiment, the composition for treating keratin fibers according to the invention comprises at least one cationic surfactant of natural origin.

In one embodiment, the composition for treating keratin fibers according to the invention comprises at least one cationic surfactant of plant origin.

In one embodiment, the composition for treating keratin fibers according to the invention comprises at least one cationic surfactant of animal origin.

In one embodiment, the composition for treating keratin fibers according to the invention comprises at least one cationic surfactant of synthetic origin.

In one embodiment, the composition for treating keratin fibers according to the invention comprises at least one cationic surfactant chosen from the group consisting of esterquats, tetraalkylammoniums, trimethylalkylammonium halides, trimethylalkylammonium methosulfates and N-[3-(dimethylamino)propyl]alkanamides.

In one embodiment, the composition for treating keratin fibers according to the invention comprises at least one cationic surfactant chosen from the group consisting of esterquats, that is to say a compound having both at least one ester function and at least one quaternary ammonium group.

In one embodiment, the esterquat cationic surfactant is selected from the group consisting of salts of quaternized fatty acid esters with triethanolamine, salts of quaternized fatty acid esters with diethanolalkylamines, and salts of quaternized fatty acid esters with 1,2-dihydroxypropyldialkylamines such as methyl-N-(2-hydroxyethyl)-N,N-di(tallowacyloxyethyl) ammonium, bis-(palmitoyloxyethyl) hydroxyethylmethyl ammonium, methyl-N,N-bis(stearoyloxyethyl)-N-(2-hydroxyethyl) ammonium, methyl-N,N-bis(cocoyloxyethyl)-N-(2-hydroxyethyl) ammonium, or N,N-dimethyl-N,N-di(tallowacyloxyethyl) ammonium, alone or as a mixture.

In one embodiment, the composition for treating keratin fibers according to the invention comprises at least one esterquat cationic surfactant chosen from the group consisting of distearoylethyl dimonium chloride, dibehenoylethyl dimonium chloride, dipalmitoylethyl dimonium chloride, ditallowoyl PG-dimonium chloride, dipalmitoylethyl hydroxyethylmonium methosulfate, distearoylethyl hydroxyethylmonium methosulfate, alone or as a mixture.

In one embodiment, the composition for treating keratin fibers according to the invention contains at least one cationic surfactant selected from the group consisting of behentrimonium chloride, cetrimonium chloride, quatemium-22, behenylamidopropyl-2,3-dihydroxypropyl-dimethylammonium chloride, palmitylamidopropyltrimethylammonium chloride, oleocetyldimethyl-hydroxyethylammonium chloride, stearamidopropyldimethyl (myristyl acetate) ammonium chloride, an ammonium salt of di(C₁-C₂ alkyl) (C₁₂-C₂₂ alkyl)hydroxy(C₁-C₂ alkyl), an alkyltrimethylammonium salt with a C₁₂-C₂₄ alkyl radical, propanetallowdiammonium dichloride, behentrimonium methosulfate, chlorides and methylsulfates of diacyloxyethyl-dimethylammonium, of diacyloxyethyl-hydroxyethyl-methylammonium, of monoacyloxyethyl-dihydroxyethyl-methyl-ammonium, triacyloxyethyl-methylammonium, or of monoacyloxyethyl-hydroxyethyl-dimethyl-ammonium, distearyldimethylammonium chloride, behentrimonium methosulfate, alone or as mixtures.

In one embodiment, the composition for treating keratin fibers according to the invention comprises at least one cationic surfactant selected from the group consisting of lauryltrimethylammonium chloride, lauryltrimethylammonium methosulfate, cetyltrimethylammonium chloride, cetyltrimethylammonium bromide, cetyltrimethylammonium methosulfate, dicetyldimethylammonium chloride, dicetyldimethylammonium methosulfate, tricetylmethylammonium chloride, tricetylmethylammonium methosulfate, stearyltrimethylammonium chloride, stearyltrimethylammonium methosulfate, distearyldimethylammonium chloride, distearyldimethylammonium methosulfate, lauryldimethylbenzylammonium chloride, behenyltrimethylammonium chloride, behenyltrimethylammonium bromide, behenyltrimethylammonium methosulfate, dimethyldibehenylammonium chloride, tribehenylmethyltrimonium chloride, cocotrimonium chloride, cocotrimonium methosulfate, dicocodimonium chloride, dicocodimonium methosulfate, tricocomonium chloride, tricocomonium methosulfate, distearoylethylhydroxyethylmonium methosulfates and dicocoylethylhydroxyethylmonium methosulfates.

In one embodiment, the composition for treating keratin fibers according to the invention comprises at least one cationic surfactant chosen from the group of fatty amines or their salts.

In one embodiment, the composition for treating keratin fibers according to the invention comprises at least one cationic surfactant chosen from the group consisting of primary fatty amines, secondary fatty amines, tertiary fatty amines and quaternary ammonium salts.

In one embodiment, the composition for treating keratin fibers according to the invention comprises at least one cationic surfactant chosen from the group consisting of polyoxyalkylenated fatty amines or their salts.

In one embodiment, the composition for treating keratin fibers according to the invention comprises at least one fatty amine surfactant carrying at least one C₈-C₃₀ hydrocarbon chain.

In one embodiment, the composition for treating keratin fibers according to the invention comprises at least one cationic surfactant chosen from the group consisting of quaternary ammonium salts.

In one embodiment, the composition for treating keratin fibers according to the invention comprises at least one cationic surfactant quaternary ammonium salt chosen from the group consisting of tetraalkylammonium salts.

In one embodiment, the composition for treating keratin fibers according to the invention comprises at least one cationic surfactant tetraalkylammonium salt chosen from the group consisting of dialkyldimethylammonium salts and alkyltrimethylammonium salts, the alkyl group being C₁₂-C₂₂.

In one embodiment, the composition for treating keratin fibers according to the invention comprises at least one cationic surfactant in the group consisting of behenyltrimethyl ammonium salts, distearyldimethyl ammonium salts, cetyltrimethyl ammonium salts, benzyldimethylstearyl ammonium salts, palmitylamidopropyltrimethyl ammonium salts, stearamidopropyltrimethyl ammonium salts, stearamidopropyldimethylcetearyl ammonium salts and stearamidopropyldimethyl-(myristylacetate) ammonium salts.

In one embodiment, the composition for treating keratin fibers according to the invention comprises at least one cationic surfactant chosen from the group consisting of quaternary ammonium salts containing at least one ester function.

In one embodiment, the composition for treating keratin fibers according to the invention comprises at least one cationic surfactant chosen from the group consisting of quaternary ammonium salts containing two ester functions.

In one embodiment, the composition for treating keratin fibers according to the invention comprises at least one cationic surfactant in the group consisting of diacyloxyethyl-dimethyl ammonium salts, diacyloxyethyl-hydroxyethyl-methyl ammonium salts, monoacyloxyethyl-dihydroxyethyl-methyl ammonium salts, triacyloxyethyl-methyl ammonium salts and monoacyloxyethyl-hydroxyethyl-dimethyl ammonium salts.

In one embodiment, the composition for treating keratin fibers according to the invention comprises at least one cationic surfactant chosen from the group consisting of quaternary ammonium salts of imidazoline.

In one embodiment, the composition for treating keratin fibers according to the invention comprises at least one neutral surfactant.

The neutral surfactant can be of plant, animal, mineral or synthetic origin.

In one embodiment, the composition for treating keratin fibers according to the invention comprises at least one neutral surfactant of natural origin.

In one embodiment, the composition for treating keratin fibers according to the invention comprises at least one neutral surfactant of plant origin.

In one embodiment, the composition for treating keratin fibers according to the invention comprises at least one neutral surfactant of animal origin.

In one embodiment, the composition for treating keratin fibers according to the invention comprises at least one neutral surfactant of synthetic origin.

In one embodiment, the composition for treating keratin fibers according to the invention comprises at least one neutral surfactant chosen from the group consisting of linear fatty alcohols comprising ethylene oxide and/or propylene oxide groups.

In one embodiment, the composition for treating keratin fibers according to the invention comprises at least one neutral surfactant chosen from the group consisting of linear fatty alcohols comprising ethylene oxide groups.

In one embodiment, the composition for treating keratin fibers according to the invention comprises at least one neutral surfactant chosen from the group consisting of linear fatty alcohols comprising propylene oxide groups.

In one embodiment, the composition for treating keratin fibers according to the invention comprises at least one neutral surfactant chosen from the group consisting of polyalkylene glycol ethers of a C₁₂-C₂₀ fatty alcohol comprising from 5 to 30 ethylene oxide groups and from 0 to 10 propylene oxide groups.

In one embodiment, the composition for treating keratin fibers according to the invention comprises at least one neutral surfactant chosen from the group consisting of polyalkylene glycol ethers of a C₁₂-C₂₀ fatty alcohol comprising from 10 to 25 ethylene oxide groups and from 0 to 6 propylene oxide groups.

In one embodiment, the composition for treating keratin fibers according to the invention comprises at least one neutral surfactant chosen from the group consisting of polyalkylene glycol ethers of a C₁₂-C₂₀ fatty alcohol comprising from 18 to 22 ethylene oxide groups and from 0 to 6 propylene oxide groups.

In one embodiment, the composition for treating keratin fibers according to the invention comprises a mixture of two neutral surfactants.

In one embodiment, the composition for treating keratin fibers according to the invention comprises a mixture of a first polyalkylene glycol ether of a fatty alcohol and a second polyalkylene glycol ether of a fatty alcohol.

In one embodiment, the composition for treating keratin fibers according to the invention comprises a mixture of a first polyalkylene glycol ether of a fatty alcohol and a second polyalkylene glycol ether of a fatty alcohol, the first polyalkylene glycol ether of a fatty alcohol comprising fewer carbon atoms than the second polyalkylene glycol ether of a fatty alcohol.

In one embodiment, the composition for treating keratin fibers according to the invention comprises at least one neutral surfactant chosen from the group consisting of compounds bearing the INCI names Steareth-2, Steareth-21, Oleth-10, PEG-100 stearates and PPG-5-ceteth-20.

In one embodiment, the composition for treating keratin fibers according to the invention comprises at least one neutral surfactant chosen from the group consisting of the addition products of 2 to 20 moles of ethylene oxide with beeswax.

In one embodiment, the composition for treating keratin fibers according to the invention comprises at least one neutral surfactant chosen from the group consisting of fatty acid and sugar esters and alkyl polyglucosides.

In one embodiment, the composition for treating keratin fibers according to the invention comprises at least one neutral surfactant chosen from the group consisting of fatty acid and sugar esters.

In one embodiment, the composition for treating keratin fibers according to the invention comprises at least one fatty acid and sugar ester chosen from the group consisting of monoesters or polyesters of fatty acid of sugar or alkyl sugar.

In one embodiment, the composition for treating keratin fibers according to the invention comprises at least one fatty acid and sugar ester chosen from the group consisting of oxyalkylenated, for example oxyethylenated and/or oxypropylenated, or polyglycerolated monoesters or polyesters of fatty acid of sugar or alkyl sugar.

In one embodiment, the composition for treating keratin fibers according to the invention comprises at least one fatty acid and sugar ester chosen from the group consisting of fatty acid esters or mixtures of C₁₁-C₂₂ fatty acid esters, optionally with one or more unsaturations, and sucrose, maltose, glucose, fructose and xylose.

In one embodiment, the composition for treating keratin fibers according to the invention comprises at least one fatty acid and sugar ester chosen from the group consisting of esters or mixtures of C₁₁-C₂₂ fatty acid esters, optionally with one or more unsaturations, and C₁-C₄ alkyl glucose such as methylglucose.

In one embodiment, the composition for treating keratin fibers according to the invention comprises at least one fatty acid and sugar ester carrying a linear or branched, saturated or unsaturated C₁₁-C₂₂, C₁₂-C₂₀, C₁₄-C₂₀ or C₁₆-C₁₈ hydrocarbon chain.

In one embodiment, the composition for treating keratin fibers according to the invention comprises at least one ester of fatty acid and sugar, the fatty unit of the esters being chosen from the group consisting of stearates, behenates, cocoates, arachidonates, palmitates, myristates, laurates, caprates and oleates.

In one embodiment, the composition for treating keratin fibers according to the invention comprises at least one ester of fatty acid and sugar, the sugar unit possibly comprising a C₁-C₄alkyl group.

In one embodiment, the composition for treating keratin fibers according to the invention comprises at least one fatty acid and sugar ester, the sugar unit being chosen from the group consisting of sucrose, maltose, glucose, fructose, mannose, galactose, arabinose, xylose, lactose, trehalose and methylglucose.

In one embodiment, the composition for treating keratin fibers according to the invention comprises at least one ester of fatty acids and sugar chosen from the group consisting of esters of sucrose and C₁₂-C₃₀ fatty acids, in particular C₁₄-C₂₀, said esters possibly comprising from 2 to 5 fatty chains.

In one embodiment, the composition for treating keratin fibers according to the invention comprises at least one ester of fatty acids and sugar chosen from the group consisting of diesters of fatty acids and sugar.

In one embodiment, the composition for treating keratin fibers according to the invention comprises at least one ester of fatty acids and sugar chosen from the group consisting of methyl glucose dioleates.

In one embodiment, the composition for treating keratin fibers according to the invention comprises at least one neutral surfactant chosen from the group consisting of alkyl polyglucosides.

In one embodiment, the composition for treating keratin fibers according to the invention comprises at least one neutral surfactant chosen from the group consisting of polyalkolxylated alkyl polyglucosides.

In one embodiment, the composition for treating keratin fibers according to the invention comprises at least one neutral surfactant chosen from the group consisting of alkyl polyglucosides of general formula RO-(G)ₘ in which R denotes a saturated or unsaturated, linear or branched, C₁₂-C₄₄ hydrocarbon chain and the group G denotes a C₅-C₆ saccharide residue and m is a number ranging from 1 to 10, preferably from 1 to 5, and even more preferably m is equal to 1.

In one embodiment, the composition for treating keratin fibers according to the invention comprises at least one alkyl polyglucoside of general formula RO-(G)ₘ in which R denotes a branched C₁₂-C₄₄ alkyl radical, preferably C₁₆-C₃₆, and better still C₁₈-C₃₀.

In one embodiment, the composition for treating keratin fibers according to the invention comprises at least one alkyl polyglucoside of general formula RO-(G)ₘ in which the saccharide residue G is chosen from the group consisting of glucose, dextrose, sucrose, fructose, galactose, maltose, maltotriose, lactose, cellobiose, mannose, ribose, dextran, talose, allose, xylose, levoglucan, cellulose, starch and methyl glucose.

In one embodiment, the composition for treating keratin fibers according to the invention comprises at least one alkyl polyglucoside chosen from the group consisting of ethers or mixtures of ethers of C₁₂-C₄₄ fatty alcohol and glucose, maltose, sucrose, xylose or fructose and ethers or mixtures of ethers of C₁₂-C₄₄ fatty alcohol and methylglucose.

In one embodiment, the composition for treating keratin fibers according to the invention comprises at least one alkyl polyglucoside carrying at least one fatty alcohol ether, the fatty unit being chosen from the group consisting of cetyl, behenyl, arachidyl, stearyl, palmityl, myristyl, lauryl, hexadecanoyl, octyldodecyl units and their mixtures such as cetearyl.

In one embodiment, the composition for treating keratin fibers according to the invention comprises at least one alkyl polyglucoside of formula SO-G in which S denotes a saturated branched alkyl radical in C₁₂-C₄₄, the group G denotes a saccharide residue in C₅-C₆, preferably a xylose residue.

In one embodiment, the composition for treating keratin fibers according to the invention comprises at least one C₆-C₃₀ alkyl polyglucoside, preferably C₈-C₁₆ alkyl, and preferably comprising 1, 2 or 3 saccharide units.

In one embodiment, the composition for treating keratin fibers according to the invention comprises at least one alkyl polyglucoside chosen from the group consisting of decylglucoside (Alkyl-C₉-C₁₁-polyglucoside (1.4)), caprylyl/capryl glucoside, laurylglucoside, coco-glucoside, cetostearyl glucoside, cetostearyl glucoside in a mixture with cetostearyl alcohol, arachidyl glucoside and cocoylethylglucoside.

In one embodiment, the composition for treating keratin fibers according to the invention comprises at least one neutral surfactant chosen from the group consisting of polyoxyethylenated sorbitol fatty esters, polyoxyethylenated fatty alcohols, polyoxyethylenated alkylphenols, ethylene oxide and propylene oxide condensates, esters of fatty acids and glycerol or polyglycerol such as, for example, glyceryl isostearate, polyglyceryl 3 diisostearate, glyceryl caprylate, polyethylene glycol and/or polypropylene glycol and glycerol ethers, esters resulting from the reaction between fatty acids and polyethylene glycol and/or polypropylene glycol glycerol ethers.

In one embodiment, the composition for treating keratin fibers according to the invention comprises at least one neutral surfactant chosen from the group consisting of glycerol esters of fatty acids.

In one embodiment, the composition for treating keratin fibers according to the invention comprises at least one neutral surfactant chosen from the group consisting of C₁₂-C₂₂ fatty acid monoesters with glycerol.

In one embodiment, the composition for treating keratin fibers according to the invention comprises at least one neutral surfactant chosen from the group consisting of glyceryl monomyristate, glyceryl palmitate, glyceryl stearate, glyceryl isostearate, glyceryl oleate, glyceryl dioleate, glyceryl distearate, glyceryl laurate, glyceryl trilaurate, triarachidin, tribehenin, tricaprin, tricaprylin, caprylic/capric triglyceride, trierucin, triheptanoin, triheptylundecanoin, triisononanoin, triisopalmitin, triisostearin, trilinolein, trimyristin, trioctanoin, triolein, tripalmitin, tripalmitolein, triricinolein, tristearin and triundecanoin.

In one embodiment, the composition for treating keratin fibers according to the invention comprises at least one neutral surfactant chosen from oligomeric glycerol esters.

In one embodiment, the composition for treating keratin fibers according to the invention comprises at least one oligomeric glycerol ester chosen from the group consisting of diglycerol esters and triglycerol esters.

In one embodiment, the composition for treating keratin fibers according to the invention comprises at least one glycerol ester selected from the group consisting of bis-diglyceryl polyacyladipate-2, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl caprylate, glyceryl stearate citrate and glyceryl stearate lactate.

In one embodiment, the composition for treating keratin fibers according to the invention comprises at least one neutral surfactant chosen from the group of polymeric neutral surfactants.

In one embodiment, the polymeric neutral surfactant is selected from the group of hydrophobic modified inulins.

In one embodiment, the composition for treating keratin fibers according to the invention comprises at least one anionic surfactant.

The neutral surfactant can be of plant, animal, mineral or synthetic origin.

In one embodiment, the composition for treating keratin fibers according to the invention comprises at least one anionic surfactant of natural origin.

In one embodiment, the composition for treating keratin fibers according to the invention comprises at least one anionic surfactant of plant origin.

In one embodiment, the composition for treating keratin fibers according to the invention comprises at least one anionic surfactant of animal origin.

In one embodiment, the composition for treating keratin fibers according to the invention comprises at least one anionic surfactant of synthetic origin.

In one embodiment, the composition for treating keratin fibers according to the invention comprises at least one anionic surfactant chosen from the group consisting of alkyl sulfates, alkyl ether sulfates and their salts, and ammonium alkyl ether sulfates.

In one embodiment, the composition for treating keratin fibers according to the invention comprises at least one anionic surfactant chosen from the group consisting of sodium lauryl ether sulfates and ammonium lauryl ether sulfates.

In one embodiment, the composition for treating keratin fibers according to the invention comprises at least one anionic surfactant chosen from the group consisting of alkyl sulfoacetates.

In one embodiment, the composition for treating keratin fibers according to the invention comprises at least one anionic surfactant chosen from the group consisting of alkyl sulfosuccinates.

In one embodiment, the composition for treating keratin fibers according to the invention comprises at least one anionic surfactant selected from the group consisting of oxyethylenated (3 EO) lauryl alcohol monosulfosuccinate (C₁₂/C₁₄, 70/30), the disodium salt of C₁₂-C₁₄ alcohols hemisulfosuccinate, oxyethylenated (2 EO) disodium oleamidosulfosuccinate, oxyethylenated (5 EO) lauryl amide monosulfosuccinate, oxyethylenated (10 EO) lauryl citrate monosulfosuccinate disodium salt and ricinoleic monoethanolamide monosulfosuccinate salt.

In one embodiment, the composition for treating keratin fibers according to the invention comprises at least one anionic surfactant chosen from the group consisting of polypeptides.

In one embodiment, the composition for treating keratin fibers according to the invention comprises at least one anionic surfactant chosen from the group consisting of polypeptides obtained by condensation reaction to functionalize a cereal amino acid chain with a fatty chain.

In one embodiment, the polypeptide-type anionic surfactant is derived from a cereal amino acid chain selected from the group consisting of wheat, oats, soybeans and copra.

In one embodiment, the polypeptide-type anionic surfactant is selected from the group consisting of the potassium salt of hydrolyzed wheat lauroyl protein, the triethanolamine salt of hydrolyzed cocoyl soy protein, the sodium salt of oat lauroyl amino acids, the hydrolyzate of collagen grafted onto copra fatty acid and soy proteins acylated by hydrogenated copra acids.

In one embodiment, the composition for treating keratin fibers according to the invention comprises at least one anionic surfactant chosen from the group consisting of amino acid derivatives.

In one embodiment, the composition for treating keratin fibers according to the invention comprises at least one anionic surfactant derived from amino acids chosen from the group consisting of sarcosinates, acylsarcosinates, alaninates, N-acylglutamates, aspartates and glycine derivatives.

In one embodiment, the composition for treating keratin fibers according to the invention comprises at least one anionic surfactant chosen from the group consisting of acylsarcosinates.

In one embodiment, the anionic acylsarcosinate surfactant is selected from the group consisting of sodium lauroyl sarcosinate, sodium myristoyl sarcosinate, and sodium palmitoyl sarcosinate.

In one embodiment, the composition for treating keratin fibers according to the invention comprises at least one anionic surfactant chosen from the group consisting of alaninates.

In one embodiment, the alaninate anionic surfactant is selected from the group consisting of sodium N-lauroyl-N-methylamidopropionate and N-lauroyl-N-methylalanine triethanolamine.

In one embodiment, the composition for treating keratin fibers according to the invention comprises at least one anionic surfactant chosen from the group consisting of N-acylglutamates.

In one embodiment, the anionic surfactant N-acylglutamate is selected from the group consisting of triethanolamine mono-cocoylglutamate and triethanolamine lauroyl-glutamate.

In one embodiment, the composition for treating keratin fibers according to the invention comprises at least one anionic surfactant chosen from the group consisting of aspartates.

In one embodiment, the anionic aspartate surfactant is selected from the group consisting of triethanolamine N-lauroylaspartate and triethanolamine N-myristoylaspartate.

In one embodiment, the composition for treating keratin fibers according to the invention comprises at least one anionic surfactant chosen from the group consisting of glycine derivatives.

In one embodiment, the glycine-derived anionic surfactant is selected from the group consisting of sodium N-cocoylglycinate and potassium N-cocoylglycinate.

In one embodiment, the composition for treating keratin fibers according to the invention comprises at least one anionic surfactant chosen from the group consisting of sulfonates.

In one embodiment, the composition for treating keratin fibers according to the invention comprises at least one anionic surfactant chosen from the group consisting of alpha-olefin sulfonates.

In one embodiment, the composition for treating keratin fibers according to the invention comprises at least one anionic surfactant chosen from the group consisting of alkyl aryl sulfonates.

In one embodiment, the alkyl aryl sulfonates anionic surfactant is sodium xylene sulfonate.

In one embodiment, the composition for treating keratin fibers according to the invention comprises at least one anionic surfactant chosen from the group consisting of isethionates.

In one embodiment, the composition for treating keratin fibers according to the invention comprises at least one anionic surfactant chosen from the group consisting of acylisethionates.

In one embodiment, the anionic acylisethionate surfactant is cocoyl isethionate.

In one embodiment, the composition for treating keratin fibers according to the invention comprises at least one zwitterionic or amphoteric surfactant.

The zwitterionic or amphoteric surfactant can be of plant, animal, mineral or synthetic origin.

In one embodiment, the composition for treating keratin fibers according to the invention comprises at least one zwitterionic or amphoteric surfactant of natural origin.

In one embodiment, the composition for treating keratin fibers according to the invention comprises at least one zwitterionic or amphoteric surfactant of plant origin.

In one embodiment, the composition for treating keratin fibers according to the invention comprises at least one zwitterionic or amphoteric surfactant of animal origin.

In one embodiment, the composition for treating keratin fibers according to the invention comprises at least one zwitterionic or amphoteric surfactant of synthetic origin.

In one embodiment, the composition for treating keratin fibers according to the invention comprises at least one zwitterionic or amphoteric surfactant chosen from the group consisting of alkylamido alkylamine derivatives, betaines, alkylamidopropyl betaines and their derivatives, and imidazoline derivatives.

In one embodiment, the composition for treating keratin fibers according to the invention comprises at least one zwitterionic or amphoteric surfactant chosen from the group consisting of alkylamido alkylamine derivatives.

In one embodiment, the alkylamido alkylamine derivative surfactant is selected from the group consisting of N-cocoyl-N-carboxymethoxyethyl-N-carboxymethyl-ethylenediamine N-disodium, N-cocoyl-N-hydroxyethyl-N-carboxymethyl-ethylenediamine N-sodium and a mixture of coconut acid ethanolamides.

In one embodiment, the composition for treating keratin fibers according to the invention comprises at least one zwitterionic or amphoteric surfactant chosen from the group consisting of imidazoline derivatives.

In one embodiment, the composition for treating keratin fibers according to the invention comprises at least one zwitterionic or amphoteric surfactant chosen from the group consisting of betaines.

In one embodiment, the composition for treating keratin fibers according to the invention comprises at least one zwitterionic or amphoteric surfactant chosen from the group consisting of alkylbetaines, alkylsulfobetaines, alkylamidoalkylbetaines and alkylamidoalkylsulfobetaines.

In one embodiment, the composition for treating keratin fibers according to the invention comprises at least one zwitterionic or amphoteric surfactant chosen from the group consisting of alkyl(C₈-C₂₀)betaines, alkyl(C₈-C₂₀)sulfobetaines, alkyl(C₈-C₂₀)amidoalkyl(C₃-C₈)betaines and alkyl(C₈-C₂₀)amidoalkyl(C₆-C₈)sulfobetaines.

In one embodiment, the zwitterionic or amphoteric betaine surfactant is selected from the group consisting of cocobetaine, laurylbetaine, oxyethylenated laurylbetaine and oxyethylenated stearylbetaine.

In one embodiment, the composition for treating keratin fibers according to the invention comprises at least one zwitterionic or amphoteric surfactant chosen from the group consisting of alkylamidopropyl betaines and their derivatives.

In one embodiment, the alkylamidopropylbetaine zwitterionic or amphoteric surfactant is selected from the group consisting of cocamidopropylbetaine and lauramidopropylbetaine.

In one embodiment, the composition for treating keratin fibers according to the invention is characterized in that the mass proportion of the at least one surfactant is between 0.001 and 1.5% relative to the total mass of the composition.

In one embodiment, the composition for treating keratin fibers according to the invention is characterized in that the mass proportion of the at least one surfactant is between 0.005 and 1% relative to the total mass of the composition.

In one embodiment, the composition for treating keratin fibers according to the invention comprises at least one cationic polymer.

In one embodiment, the composition for treating keratin fibers according to the invention comprises at least one cationic polymer of plant and/or animal origin.

In one embodiment, the composition for treating keratin fibers according to the invention comprises at least one cationic polymer of plant origin.

In one embodiment, the composition for treating keratin fibers according to the invention comprises at least one cationic polymer of animal origin.

In one embodiment, the composition for treating keratin fibers according to the invention comprises at least one cationic polymer derived from a polysaccharide.

In one embodiment, the composition for treating keratin fibers according to the invention comprises at least one cationic polymer derived from a natural polysaccharide.

In one embodiment, the composition for treating keratin fibers according to the invention comprises at least one cationic polymer derived from a synthetic polysaccharide.

In one embodiment, the composition for treating keratin fibers according to the invention comprises at least one cationic polymer chosen from gums.

In one embodiment, the composition for treating keratin fibers according to the invention comprises at least one cationic polymer selected from the group of gums consisting of acacia, agar, alin, alginic acid, ammonium alginate, potassium alginate, calcium alginate, propylene glycol alginate, amylopectin, calcium carrageenan, sodium carrageenan, potassium carrageenan, carrageenan, dextran, sodium carboxymethyldextran, gelatin, gellan gum, guar gum, hydroxypropyl guar, guar hydroxypropyltrimonium chloride, hydroxypropyl guar hydroxypropyltrimonium chloride, karaya gum, locust bean gum, natto gum, sclerotium gum, tragacanth gum and gum of xanthan.

In one embodiment, the composition for treating keratin fibers according to the invention comprises at least one cationic polymer chosen from the group of cationic cellulosic polysaccharides.

In one embodiment, the composition for treating keratin fibers according to the invention comprises at least one cationic polymer chosen from the group of cationic polysaccharides consisting of cellulose ether derivatives bearing quaternary ammonium groups, cationic cellulose copolymers, cellulose derivatives grafted with a water-soluble quaternary ammonium monomer and cationic galactomannan gums.

In one embodiment, the composition for treating keratin fibers according to the invention comprises at least one cationic polymer chosen from the group consisting of associative celluloses.

In one embodiment, the composition for treating keratin fibers according to the invention comprises at least one cationic polymer of associative cellulose type chosen from the group consisting of quaternized alkylhydroxyethylcelluloses with C₈-C₃₀ fatty chains.

In one embodiment, the composition for treating keratin fibers according to the invention comprises at least one cationic polymer chosen from non-cellulosic cationic polysaccharides.

In one embodiment, the composition for treating keratin fibers according to the invention comprises at least one cationic polymer chosen from the group of cationic proteins or cationic protein hydrolysates.

In one embodiment, the composition for treating keratin fibers according to the invention comprises at least one cationic protein polymer or protein hydrolyzate chosen from the group of polypeptides chemically modified by incorporation at the end of the chain or grafting onto the latter of one or more quaternary ammonium groups.

In one embodiment, the composition for treating keratin fibers according to the invention comprises at least one cationic polymer of polypeptide type chosen from the group of modified collagen hydrolysates carrying one or more quaternary ammonium groups.

In one embodiment, the composition for treating keratin fibers according to the invention comprises at least one cationic polymer of polypeptide type chosen from the group of modified animal protein hydrolysates carrying one or more quaternary ammonium groups.

In one embodiment, the composition for treating keratin fibers according to the invention comprises at least one cationic polymer chosen from the group of quaternized plant proteins.

In one embodiment, the composition for treating keratin fibers according to the invention comprises at least one cationic polymer chosen from the group of wheat proteins, corn proteins and quaternized soy proteins.

In one embodiment, the composition for treating keratin fibers according to the invention comprises at least one non-cellulosic cationic polysaccharide chosen from guar gums carrying cationic groups of trialkylammonium type.

In one embodiment, the composition for treating keratin fibers according to the invention comprises at least one cationic polymer chosen from the group of homopolymers and copolymers obtained by the polymerization of monomers of acrylamidoalkyltrialkylammonium type.

In one embodiment, the composition for treating keratin fibers according to the invention comprises at least one cationic polymer chosen from the group of polymers obtained by the polymerization of acrylamidopropyltrimethylammonium chloride (APTAC).

In one embodiment, the composition for treating keratin fibers according to the invention comprises at least one cationic polymer chosen from the group consisting of polymers carrying primary, secondary, tertiary and/or quaternary amine groups present either in the main chain of the polymer or in the side chains of the polymer directly linked to it.

In one embodiment, the composition for treating keratin fibers according to the invention comprises at least one cationic polymer chosen from the group of polymers of polyamine, polyaminoamide and polyquaternary ammonium type.

In one embodiment, the composition for treating keratin fibers according to the invention comprises at least one cationic polymer chosen from the group of copolymers obtained by polymerization of at least two monomers of the acrylamide, methacrylamide, diacetone acrylamide, acrylic acid, methacrylic acid, acrylic acid ester, methacrylic acid ester, vinyllactam, vinylpyrrolidone, vinylcaprolactam and vinyl ester type.

In one embodiment, the composition for treating keratin fibers according to the invention comprises at least one cationic polymer of the water-soluble polyaminoamide type prepared in particular by polylycondensation of an acidic compound with a polyamine.

In one embodiment, the composition for treating keratin fibers according to the invention comprises at least one cationic polymer of the water-soluble and crosslinked polyaminoamide type.

In one embodiment, the composition for treating keratin fibers according to the invention comprises at least one non-ionic or neutral polymer.

In one embodiment, the composition for treating keratin fibers according to the invention comprises at least one neutral polymer of natural origin.

In one embodiment, the composition for treating keratin fibers according to the invention comprises at least one neutral polymer of synthetic origin.

In one embodiment, the composition for treating keratin fibers according to the invention comprises at least one neutral polymer of aliphatic ester type resulting from the esterification reaction between an aliphatic ester of hydroxycarboxylic acid and an aliphatic carboxylic acid.

In one embodiment, the composition for treating keratin fibers according to the invention comprises at least one neutral polymer of non-crosslinked polyester type resulting from a polycondensation reaction between linear or branched C₄-C₅₀ dicarboxylic acid or polycarboxylic acid and a C₂-C₅₀ diol or polyol.

In one embodiment, the composition for treating keratin fibers according to the invention comprises at least one anionic polymer.

In one embodiment, the composition for treating keratin fibers according to the invention comprises at least one anionic polymer of natural origin.

In one embodiment, the composition for treating keratin fibers according to the invention comprises at least one anionic polymer of synthetic origin.

In one embodiment, the composition for treating keratin fibers according to the invention comprises at least one anionic polymer chosen from the group of copolymers of acrylic acid and methacrylic acid.

In one embodiment, the composition for treating keratin fibers according to the invention comprises at least one anionic polymer chosen from the group of copolymers of acrylic acid and methacrylic acid modified by at least one hydrophobic group.

In one embodiment, the composition for treating keratin fibers according to the invention comprises at least one anionic polymer chosen from the group of copolymers of acrylic acid and methacrylic acid modified by at least one hydrophobic group and being crosslinked.

In one embodiment, the composition for treating keratin fibers according to the invention comprises at least one anionic polymer chosen from the group of copolymers of acrylic acid and methacrylic acid and copolymers of acrylic acid and methacrylic acid modified by at least one hydrophobic group.

In one embodiment, the composition for treating keratin fibers according to the invention comprises at least one anionic polymer chosen from the group of copolymers of acrylic acid and methacrylic acid and copolymers of acrylic acid and methacrylic acid modified by at least one hydrophobic group and being crosslinked.

In one embodiment, the composition for treating keratin fibers according to the invention comprises at least one anionic polymer chosen from the group of acrylic acid/methacrylic acid and C₁₀-C₃₀ alkyl acrylate/methacrylate copolymers.

In one embodiment, the composition for treating keratin fibers according to the invention comprises at least one anionic polymer chosen from the group of copolymers of acrylic acid/methacrylic acid and of C₁₀-C₃₀ alkyl acrylate/methacrylate being crosslinked.

In one embodiment, the composition for treating keratin fibers according to the invention comprises at least one fatty alcohol.

In one embodiment, the composition for treating keratin fibers according to the invention comprises at least one fatty alcohol of natural origin.

In one embodiment, the composition for treating keratin fibers according to the invention comprises at least one fatty alcohol of plant and/or animal origin.

In one embodiment, the composition for treating keratin fibers according to the invention comprises at least one fatty alcohol of plant origin.

In one embodiment, the composition for treating keratin fibers according to the invention comprises at least one fatty alcohol of animal origin.

In one embodiment, the composition for treating keratin fibers according to the invention comprises at least one fatty alcohol chosen from oxyalkyl fatty alcohols and fatty alcohols derived from glycerol.

In one embodiment, the composition for treating keratin fibers according to the invention comprises at least one fatty alcohol represented by the formula R—OH, in which R denotes a saturated or unsaturated, linear or branched C₈-C₄₀ group, optionally substituted by one or more hydroxyl groups.

In one embodiment, R is C₁₀-C₃₀.

In one embodiment, R is C₁₂-C₂₄.

In one embodiment, R is C₁₄-C₂₂.

In one embodiment, the composition for treating keratin fibers according to the invention comprises at least one fatty alcohol chosen from the group of fatty alcohols liquid at 25° C.

In one embodiment, the composition for treating keratin fibers according to the invention comprises at least one liquid fatty alcohol chosen from the group consisting of C₁₀-C₃₄ alcohols, branched and optionally carrying one to three unsaturated bonds.

In one embodiment, the composition for treating keratin fibers according to the invention comprises at least one liquid fatty alcohol chosen from the group consisting of C₁₂-C₂₄ alcohols, branched and carrying at least one unsaturated bond.

In one embodiment, the liquid fatty alcohol is a saturated branched alcohol.

In one embodiment, the composition for treating keratin fibers according to the invention comprises at least one liquid fatty alcohol selected from the group consisting of oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-octyl-1-dodecanol, 2-decyl-1-tetradecanol, 2-undecylpentadecanol and 2-tetradecyl-1-cetanol.

In one embodiment, the composition for treating keratin fibers according to the invention comprises at least one fatty alcohol which is solid at room temperature (25° C.) and at atmospheric pressure (1.013×10⁵ Pa), this fatty alcohol being insoluble in water.

In one embodiment, the composition for treating keratin fibers according to the invention comprises at least one solid fatty alcohol chosen from the group consisting of lauryl alcohol, myristyl alcohol, cetyl alcohol, stearyl alcohol, arachidyl alcohol, behenyl alcohol, lignoceryl alcohol, ceryl alcohol, montanyl alcohol and myricyl alcohol.

In one embodiment, the composition for treating keratin fibers according to the invention is characterized in that the mass proportion of at least one fatty alcohol is between 0.001 and 20% relative to the total mass of the composition.

In one embodiment, the composition for treating keratin fibers according to the invention is characterized in that the mass proportion of at least one fatty alcohol is between 0.01 and 15% relative to the total mass of the composition.

In one embodiment, the composition for treating keratin fibers according to the invention is characterized in that the mass proportion of at least one fatty alcohol is between 0.05 and 10% relative to the total mass of the composition.

In one embodiment, the composition for treating keratin fibers according to the invention is characterized in that the mass proportion of at least one fatty alcohol is between 0.1 and 5% relative to the total mass of the composition.

In one embodiment, the composition for treating keratin fibers according to the invention is characterized in that the mass proportion of at least one fatty alcohol is between 0.1 and 3% relative to the total mass of the composition.

In one embodiment, the composition for treating keratin fibers according to the invention comprises at least one cosmetic adjuvant or additive traditionally present in formulations for treating keratin fibers, hair in particular.

In one embodiment, the composition for treating keratin fibers according to the invention comprises at least one cosmetic adjuvant or additive selected from the group consisting of gelling agents, preservatives, bactericides, dyes, softening agents, buffers, humectants, UV filters or sun filters, electrolytes, pH adjusters, anti-dandruff agents, anti-seborrheic agents, anti-hair loss agents, agents promoting hair regrowth, vitamins, pro-vitamins, mineral or organic pigments, sequestering agents, plasticizing agents, solubilizing agents, acidifying agents, mineral or organic thickening agents, polymeric thickening agents, opacifying or pearlescent agents, antioxidant agents, hydroxy acids, pigments and ceramides.

In one embodiment, the composition for treating keratin fibers according to the invention is characterized in that the mass proportion of at least one conventional cosmetic additive is between 0 and 20% relative to the total mass of the composition.

In one embodiment, the composition for treating keratin fibers according to the invention is characterized in that the mass proportion of at least one conventional cosmetic additive is between 0 and 10% relative to the total mass of the composition.

In one embodiment, the composition for treating keratin fibers according to the invention is characterized in that the mass proportion of at least one conventional cosmetic additive is between 0 and 5% relative to the total mass of the composition.

In one embodiment, the composition for treating keratin fibers according to the invention comprises at least one gelling agent.

In one embodiment, the composition for treating keratin fibers according to the invention comprises at least one gelling agent of natural origin.

In one embodiment, the composition for treating keratin fibers according to the invention comprises at least one gelling agent of plant origin.

In one embodiment, the composition for treating keratin fibers according to the invention comprises at least one gelling agent of animal origin.

In one embodiment, the composition for treating keratin fibers according to the invention comprises at least one gelling agent of synthetic origin.

In one embodiment, the composition for treating keratin fibers according to the invention comprises at least one hydrophilic gelling agent.

In one embodiment, the composition for treating keratin fibers according to the invention comprises at least one hydrophilic gelling agent chosen from the group consisting of carbovinyl polymers, modified carbovinyl polymers, polyacrylamides, homopolymers and copolymers of 2-acrylamido 2-methylpropane sulfonic acid optionally crosslinked and/or neutralized, biopolymers and polysaccharide biopolymers.

In one embodiment, the composition for treating keratin fibers according to the invention comprises at least one hydrophilic gelling agent chosen from the group consisting of carbovinyl polymers and modified carbovinyl polymers.

In one embodiment, the composition for treating keratin fibers according to the invention comprises at least one hydrophilic gelling agent chosen from the group consisting of polyacrylamides.

In one embodiment, the composition for treating keratin fibers according to the invention comprises at least one hydrophilic gelling agent chosen from the group consisting of homopolymers and copolymers of 2-acrylamido 2-methylpropane sulfonic acid, optionally crosslinked and/or neutralized.

In one embodiment, the composition for treating keratin fibers according to the invention comprises at least one gelling agent from a natural source selected from the group consisting of karaya gum, carob gum, natto gum, agar, gum arabic, guar gum, guar hydroxypropyltrimonium chloride, hydroxypropyl guar, sclerotium gum, tragacanth gum, kelp, hectorite and gelatin.

In one embodiment, the composition for treating keratin fibers according to the invention comprises at least one hydrophilic gelling agent chosen from the group consisting of biopolymers.

In one embodiment, the composition for treating keratin fibers according to the invention comprises at least one hydrophilic gelling agent chosen from the group consisting of biopolymers derived from natural sources.

In one embodiment, the composition for treating keratin fibers according to the invention comprises at least one hydrophilic gelling agent chosen from the group consisting of polysaccharide biopolymers.

In one embodiment, the polysaccharide biopolymer gelling agent is selected from the group consisting of xanthan gum, guar gum, sodium alginate, ammonium alginate, amylopectin, calcium alginate, potassium alginate, propylene glycol alginate, hyaluronic acid, hydroxypropyl chitosan, sodium carboxymethyldextran, carrageenan, calcium carrageenan, sodium carrageenan, potassium carrageenan, gellan gum, cellulose and cellulose derivatives.

In one embodiment, the polysaccharide biopolymer gelling agent is selected from the group consisting of alkylhydroxyalkylcellulose ethers.

In one embodiment, the polysaccharide biopolymer gelling agent is selected from the group of cellulose and cellulose derivatives consisting of cellulose sodium sulfate, microcrystalline cellulose, hydroxyethylcarboxymethyl cellulose, cellulose acetate propionate carboxylate, hydroxyethyl cellulose, hydroxypropyl cellulose, hydroxypropyl methyl cellulose and cetyl hydroxyethyl cellulose.

In one embodiment, the composition for treating keratin fibers according to the invention comprises at least one lipophilic gelling agent.

In one embodiment, the composition for treating keratin fibers according to the invention comprises at least one lipophilic gelling agent chosen from the group consisting of alkene copolymers.

In one embodiment, the composition for treating keratin fibers according to the invention comprises at least one lipophilic gelling agent chosen from the group consisting of “diblock” alkene copolymers, “triblock” alkene copolymers and “radial” alkene copolymers.

In one embodiment, the composition for treating keratin fibers according to the invention comprises at least one lipophilic gelling agent alkene copolymer of “diblock”, “triblock” or “radial” structure chosen from the group consisting of polystyrene/polyisoprene, polystyrene/polybutadiene, polystyrene/copoly(ethylene-butylene) copolymers, mixtures of butylene/ethylene/styrene triblock copolymer and ethylene/propylene/styrene star copolymer.

In one embodiment, the composition for treating keratin fibers according to the invention comprises at least one gelling agent chosen from the group consisting of polyacrylamide polymers.

In one embodiment, the composition for treating keratin fibers according to the invention comprises at least one gelling agent chosen from the group consisting of substituted, branched or unbranched nonionic polyacrylamide polymers.

In one embodiment, the composition for treating keratin fibers according to the invention comprises at least one gelling agent chosen from the group consisting of hetero-copolymers of acrylamides and acrylamides substituted by acrylic acids and substituted acrylic acids.

In one embodiment, the composition for treating keratin fibers according to the invention comprises at least one gelling agent chosen from the group consisting of crosslinked polyacrylate polymers.

In one embodiment, the composition for treating keratin fibers according to the invention comprises at least one gelling agent chosen from the group consisting of crosslinked polymers obtained by polymerization of one or more monomers of acrylic acid, derivatives of acrylic acid and crosslinking with a polyalkene derived from a polyol.

In one embodiment, the composition for treating keratin fibers according to the invention is characterized in that the mass proportion of the at least one gelling agent is between 0.05 and 20% relative to the total mass of the composition.

In one embodiment, the composition for treating keratin fibers according to the invention is characterized in that the mass proportion of the at least one gelling agent is between 0.1 and 10% relative to the total mass of the composition.

In one embodiment, the composition for treating keratin fibers according to the invention is characterized in that the mass proportion of the at least one gelling agent is between 0.5 and 5% relative to the total mass of the composition.

In one embodiment, the composition for treating keratin fibers according to the invention comprises at least one preservative agent.

In one embodiment, the composition for treating keratin fibers according to the invention comprises at least one preservative agent chosen from the group consisting of parabens, phenoxyethanol, chlorhexidine gluconate and polyhexamethylene biguanide hydrochloride.

In one embodiment, the composition for treating keratin fibers according to the invention comprises at least one antioxidant agent or free radical scavenger agent.

In one embodiment, the composition for treating keratin fibers according to the invention comprises at least one antioxidant agent chosen from the group consisting of ascorbic acid (vitamin C) and ascorbic acid derivatives.

In one embodiment, the composition for treating keratin fibers according to the invention comprises at least one antioxidant agent chosen from the group consisting of ascorbic acid and its salts, fatty acid esters of ascorbic acid, magnesium ascorbyl phosphate, sodium ascorbyl phosphate and ascorbyl sorbate.

In one embodiment, the composition for treating keratin fibers according to the invention comprises at least one antioxidant agent chosen from the group consisting of tocopherol (vitamin E) and tocopherol derivatives.

In one embodiment, the composition for treating keratin fibers according to the invention comprises at least one antioxidant agent chosen from the group consisting of tocopherol, tocopherol sorbate, tocopherol acetate and tocopherol esters.

In one embodiment, the composition for treating keratin fibers according to the invention comprises at least one antioxidant agent selected from the group consisting of butylated hydroxybenzoic acids and their salts, peroxides including hydrogen peroxide and perborate, thioglycolates, persulfate salts, 6-hydroxy-2,5,7,8-tetramethylchroman-2-carboxylic acid, gallic acid, alkyl esters of gallic acid, propyl gallate, uric acid, uric acid salts, alkyl esters of uric acid, sorbic acid, sorbic acid salts, lipoic acid, amines, N,N-diethylhydroxylamine, amino-1-guanidine, sulfhydryl compounds, glutathione, dihydroxyfumaric acid, acid salts dihydroxyfumaric acid, lysine pidolate, amino acids, silymarin, lysine, 1-methionine, proline, superoxide dismutase, olive extracts, tea extracts, polyphenols, pine bark proanthocyanidin, carotenoids, curcumin compounds, tetrahydrocurcumin, L-2-oxo-4-thiazolidine carboxylic acid (OCTA), melanin, rosemary extracts and grape skin/seed extracts.

In one embodiment, the composition for treating keratin fibers according to the invention is characterized in that the mass proportion of the at least one antioxidant agent is between 0.01 and 10% relative to the total mass of the composition.

In one embodiment, the composition for treating keratin fibers according to the invention is characterized in that the mass proportion of the at least one antioxidant agent is between 0.01 and 5% relative to the total mass of the composition.

In one embodiment, the composition for treating keratin fibers according to the invention is characterized in that the mass proportion of the at least one antioxidant agent is between 0.01 and 2% relative to the total mass of the composition.

In one embodiment, the composition for treating keratin fibers according to the invention is characterized in that the mass proportion of the at least one antioxidant agent is between 0.01 and 1% relative to the total mass of the composition.

In one embodiment, the composition for treating keratin fibers according to the invention comprises at least one softening agent.

In one embodiment, the composition for treating keratin fibers according to the invention comprises at least one softening agent chosen from the group consisting of allantoin, bisabolol and planktons.

In one embodiment, the composition for treating keratin fibers according to the invention comprises at least one softening agent chosen from the group consisting of plant extracts.

In one embodiment, the plant-extracted softening agent is selected from the group consisting of rose extracts and sweet clover extracts.

In one embodiment, the composition for treating keratin fibers according to the invention comprises at least one electrolyte.

In one embodiment, the composition for treating keratin fibers according to the invention comprises at least one electrolyte chosen from the group consisting of disodium EDTA, sodium chloride, magnesium sulfate, monopotassium phosphate and dipotassium phosphate.

In one embodiment, the composition for treating keratin fibers according to the invention comprises at least one dye.

In one embodiment, the composition for treating keratin fibers according to the invention comprises at least one dye chosen from the group consisting of oxidation dye precursors and direct-acting dyes.

In one embodiment, the composition for treating keratin fibers according to the invention comprises at least one dye chosen from the group consisting of oxidation dye precursors.

In one embodiment, the composition for treating keratin fibers according to the invention comprises at least one coloring precursor chosen from the group of components called developers.

In one embodiment, the composition for treating keratin fibers according to the invention comprises at least one coloring precursor chosen from the group of components called couplers.

A developer component can generate a color by itself while a coupler component requires the presence of a developer component to generate a coloring.

In one embodiment, the composition for treating keratin fibers according to the invention comprises at least one developer-type coloring precursor selected from the group consisting of p-phenylenediamine, p-tolylenediamine, 2-(2-hydroxyethyl)-p-phenylenediamine, 2-(1,2)-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diamino-propan-2-ol, bis-(2-hydroxy-5-aminophenyl)methane, 1,3-bis-(2,5-diaminophenoxy)-propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine and 2,3-diamino-6,7-dihydro-1H,5H-pyrazolo[1,2-a]pyrazol-1-one.

In one embodiment, the composition for treating keratin fibers according to the invention comprises at least one developer-type coloring precursor chosen from the group consisting of p-tolylenediamine, 2-(2-hydroxyethyl)-p-phenylenediamine, N,N-bis-(2-hydroxyethyl)-p-phenylenediamine, 2-methoxymethyl-p-phenylenediamine and 4,5-diamino-1-(2-hydroxyethyl)pyrazole.

In one embodiment, the composition for treating keratin fibers according to the invention comprises at least one coloring precursor of the coupler type chosen from the group consisting of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethyl-amino)benzene, 2-amino-4-[(2-hydroxyethyl)amino]-anisole, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenyl-amine, 1-amino-3-bis-(2-hydroxyethyl)aminobenzene, resorcinol, 2-methylresorcinol, 4-chlororesorcinol, 1,2,4-benzenetriol, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, naphth-1-ol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole and 7-hydroxyindole.

In one embodiment, the composition for treating keratin fibers according to the invention comprises at least one coupler-type coloring precursor chosen from the group consisting of 3-aminophenol, 5-amino-2-methylphenol, 3-amino-2-chloro-6-methylphenol, 2-hydroxy-4-aminophenoxyethanol, 2-(2,4-diaminophenoxy)ethanol, 1-methoxy-2-amino-4-(2-hydroxyethylamino)benzene, resorcinol, 2-methylresorcinol, 4-chlororesorcinol and 2-amino-3-hydroxypyridine.

In one embodiment, the composition for treating keratin fibers according to the invention comprises at least one alkalizing agent.

In one embodiment, the composition for treating keratin fibers according to the invention comprises at least one alkalizing agent chosen from the group consisting of ammonium hydroxide, basic amino acids, alkali hydroxides, alkanolamines, alkali metal metasilicates, alkali phosphates and alkali hydrogen phosphates.

In one embodiment, the composition for treating keratin fibers comprises at least one alkalizing agent selected from the group consisting of arginine, sodium hydroxide, potassium hydroxide, ammonium hydroxide and monoethanolamine.

In one embodiment, the composition for treating keratin fibers according to the invention comprises at least one direct-acting dye.

In one embodiment, the composition for treating keratin fibers according to the invention comprises at least one direct-acting dye chosen from the group consisting of nitrophenylenediamines, nitroaminophenols, azo dyes, anthraquinones and indophenols.

In one embodiment, the composition for treating keratin fibers according to the invention comprises at least one direct-acting dye chosen from the group of non-ionic direct-acting dyes.

In one embodiment, the composition for treating keratin fibers according to the invention comprises at least one nonionic direct-acting dye selected from the group consisting of HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrobenzene, 1,4-bis-(2-hydroxyethyl)-amino-2-nitrobenzene, 3-nitro-4-(2-hydroxyethyl)-aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)-amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid, 2-amino-6-chloro-4-nitrophenol, 4-ethylamino-3-nitrobenzoic acid and 2-chloro-6-ethylamino-4-nitrophenol.

In one embodiment, the composition for treating keratin fibers according to the invention comprises at least one direct-acting dye chosen from the group of cationic direct-acting dyes.

In one embodiment, the composition for treating keratin fibers according to the invention comprises at least one cationic direct-acting dye chosen from the group consisting of triphenylmethane-based compounds and aromatic compounds substituted with a quaternary nitrogen group.

In one embodiment, the composition for treating keratin fibers according to the invention comprises at least one cationic direct-acting dye chosen from the group consisting of Basic Blue 7, Basic Blue 26, Basic Violet 2, Basic Violet 14, Basic Yellow 57, Basic Red 76, Basic Blue 99, Basic Brown 16, Basic Brown 17, HC Blue 16, Basic Yellow 87, Basic Orange 31 and Basic Red 51.

In one embodiment, the composition for treating keratin fibers according to the invention comprises at least one direct-acting dye chosen from the group of anionic direct-acting dyes.

In one embodiment, the composition for treating keratin fibers according to the invention comprises at least one anionic direct-acting dye chosen from the group consisting of Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52 and tetrabromophenol blue.

In one embodiment, the composition for treating keratin fibers according to the invention is characterized in that the mass proportion of the at least one direct-acting dye is between 0.001 and 10% relative to the total mass of the composition.

In one embodiment, the composition for treating keratin fibers according to the invention is characterized in that the mass proportion of the at least one direct-acting dye is between 0.01 and 5% relative to the total mass of the composition.

In one embodiment, the composition for treating keratin fibers according to the invention is characterized in that the mass proportion of the at least one direct-acting dye is between 0.1 and 2% relative to the total mass of the composition.

In one embodiment, the composition for treating keratin fibers according to the invention comprises at least one humectant, hydrating or conditioning agent.

In one embodiment, the composition for treating keratin fibers according to the invention comprises at least one humectant.

In one embodiment, the composition for treating keratin fibers according to the invention comprises at least one moisturizing agent.

In one embodiment, the composition for treating keratin fibers according to the invention comprises at least one conditioning agent.

In one embodiment, the composition for treating keratin fibers according to the invention comprises at least one humectant, moisturizer or conditioning agent selected from the group consisting of urea, guanidine, glycolic acid, gluconate salts, salicylic acid, lactic acid, lactate salts, aloe vera, polyols, polyethylene glycols, sugars, starches, sugar derivatives, starch derivatives, hyaluronic acid, monoethanolamine lactamide, monoethanolamine acetamide, panthenol and allantoin.

In one embodiment, the composition for treating keratin fibers according to the invention comprises at least one polyol humectant, moisturizer or conditioning agent chosen from the group consisting of sorbitol, mannitol, xylitol, erythritol, glycerol, hexanetriol, butanetriol, propylene glycol, butylene glycol and hexylene glycol.

In one embodiment, the composition for treating keratin fibers according to the invention comprises at least one humectant, hydrating or conditioning agent chosen from the group consisting of sugar monoesters and polyesters.

In one embodiment, the composition for treating keratin fibers according to the invention comprises at least one humectant, hydrating or conditioning agent chosen from the group consisting of polyol monoesters and polyesters.

In one embodiment, the composition for treating keratin fibers according to the invention contains at least one conditioning agent chosen from the group consisting of urea, guanidine, sucrose polyester, panthenol, dexpanthenol, allantoin and glycerol.

In one embodiment, the composition for treating keratin fibers according to the invention comprises at least one humectant chosen from the group consisting of polyols and water-soluble alkoxylated nonionic polymers.

In one embodiment, the composition for treating keratin fibers comprises at least one humectant selected from the group consisting of glycerin, diglycerin, hexylene glycol, ethoxylated glucose, 1,2-hexanediol, maltitol, maltose, glucose, fructose, sodium chondroitin sulfate, sodium hyaluronate, sodium adenosine phosphate, sodium lactate and glucosamine.

In one embodiment, the composition for treating keratin fibers according to the invention contains at least one humectant of the water-soluble alkoxylated nonionic polymer type chosen from the group consisting of polyethylene glycols and polypropylene glycols with a weight-average molar mass (Mw) of less than or equal to 1000 g/mol.

In one embodiment, the composition for treating keratin fibers according to the invention is characterized in that the mass proportion of the at least one humectant, hydrating or conditioning agent is between 0.01 and 20% relative to the total mass of the composition.

In one embodiment, the composition for treating keratin fibers according to the invention is characterized in that the mass proportion of the at least one humectant, hydrating or conditioning agent is between 0.1 and 10% relative to the total mass of the composition.

In one embodiment, the composition for treating keratin fibers according to the invention is characterized in that the mass proportion of the at least one humectant, hydrating or conditioning agent is between 0.5 and 7% relative to the total mass of the composition.

In one embodiment, the composition for treating keratin fibers according to the invention comprises at least one sunscreen.

In one embodiment, the composition for treating keratin fibers according to the invention comprises at least one organic sunscreen.

In one embodiment, the composition for treating keratin fibers according to the invention comprises at least one inorganic sunscreen.

In one embodiment, the composition for treating keratin fibers according to the invention comprises at least one organic sunscreen active in UV-A and/or UV-B.

In one embodiment, the composition for treating keratin fibers according to the invention comprises at least one organic sunscreen selected from the group consisting of para-aminobenzoic acid derivatives, salicylic derivatives, dibenzoylmethane derivatives, cinnamic derivatives, ‘-diphenylacrylate derivatives, benzophenone derivatives, benzylidene camphor derivatives, phenylbenzimidazole derivatives, triazine derivatives, phenylbenzotriazole derivatives, imidazoline derivatives, dihydroxycinnamic acid derivatives, trihydroxycinnamic acid derivatives, hydrocarbons (diphenylbutadiene, stilbene), dibenzalacetone, benzalacetophenone, naphtholsulfonates, dihydroxynaphthoic acid and its salts, coumarin derivatives, diazoles, quinine salts, quinoline derivatives, uric acid, violuric acid, tannic acid and its derivatives, and hydroquinone.

In one embodiment, the composition for treating keratin fibers according to the invention comprises at least one organic sunscreen selected from the group consisting of 2-ethylhexyl salicylate, butyl methoxydibenzoylmethane, ethylhexyl methoxycinnamate, octocrylene, phenylbenzimidazole sulfonic acid, terephthalylidene dicamphor sulfonic acid, benzophenone-3 (oxybenzone), benzophenone-4, benzophenone-5, 4-methylbenzylidene camphor, benzimidazole, anisotriazine, ethylhexyl triazone, diethylhexyl butamido triazone, methylene bis-benzotriazolyl tetramethylbutylphenol and drometrizole trisiloxane.

In one embodiment, the composition for treating keratin fibers according to comprises at least one inorganic sunscreen chosen from the group consisting of coated or uncoated metal oxide nanopigments.

In one embodiment, the composition for treating keratin fibers according to comprises at least one inorganic sunscreen chosen from the group consisting of nanopigments coated or not with titanium oxide, iron oxide, zinc oxide, zirconium oxide and cerium oxide.

In one embodiment, the composition for treating keratin fibers according to comprises at least one inorganic sunscreen selected from the group consisting of nanopigments coated with alumina and/or aluminum stearate.

In one embodiment, the composition for treating keratin fibers according to the invention is characterized in that the mass proportion of the at least one sunscreen is between 0.1 and 20% relative to the total mass of the composition.

In one embodiment, the composition for treating keratin fibers according to the invention is characterized in that the mass proportion of the at least one sunscreen is between 0.5 and 10% relative to the total mass of the composition.

In one embodiment, the composition for treating keratin fibers according to the invention is characterized in that the mass proportion of the at least one sunscreen is between 1 and 5% relative to the total mass of the composition.

In one embodiment, the composition for treating keratin fibers according to the invention comprises at least one phase-shifting agent present in the hydrophilic phase.

In one embodiment, the composition for treating keratin fibers according to the invention comprises at least one phase-shifting agent present in the hydrophilic phase in association with a surfactant.

In one embodiment, the composition for treating keratin fibers according to the invention comprises at least one phase-separating agent chosen from the group consisting of alkylmethylbenzylammonium chlorides, alkoxylated alkyl glucosides comprising a quaternary ammonium, polyvinylpyrrolidone polymers and polyvinylpyrrolidone copolymers.

In one embodiment, the composition for treating keratin fibers according to the invention comprises at least one phase-shifting agent chosen from the group consisting of lauryl methyl gluceth-10 hydroxypropyldimonium, benzalkonium chloride and a polyvinylpyrrolidone/hexadecene copolymer.

In one embodiment, the composition for treating keratin fibers according to the invention comprises at least one chelating agent.

In one embodiment, the composition for treating keratin fibers according to the invention comprises at least one chelating agent chosen from the group consisting of carboxylic acids, polyoxycarboxylic acids, aminocarboxylic acids, amino acids, phosphoric acids, polyamines, polyphosphoric acids, polyethyleneimines, polyfunctional substituted aromatic compounds and their derivatives.

In one embodiment, the composition for treating keratin fibers according to the invention comprises at least one chelating agent selected from the group consisting of ethylenediaminetetraacetic acid (EDTA), ethylenediaminetriacetic acid, ethylenediamine-N,N′-disuccinic acid (EDDS), ethylenediamine-N,N′-diglutaric acid (EDDG), salicylic acid, aspartic acid, glutamic acid, glycine, malonic acid, histidine, sodium diethylenetriaminepentaacetate (DTPA), sodium N-hydroxyethylethylenediaminetriacetate, nitrilotriacetic acid (NTA), ethylenediaminetetrapropionate, triethylenetetraaminehexaacetate, ethanoldiglycine, propylenediaminetetraacetic acid (PDTA), methylglycine diacetic acid (MODA), diethylenetriaminepentaacetic acid, methylglycine diacetic acid (MGDA), N-acyl-N,N′,N′-ethylenediaminetriacetic acid, ethylenediaminediglutaric acid (EDGA), 2-hydroxypropylenediaminedisuccinic acid (HPDS), glycinamide-N,N′-disuccinic acid (GADS), 2-hydroxypropylenediamine-NN′-disuccinic acid (HPDDS), N-2-hydroxyethyl-N,N-diacetic acid, glyceryliminodiacetic acid, iminodiacetic acid-N-2-hydroxypropylsulfonic acid, N-carboxymethyl-N-2-hydroxypropyl-3-sulfonic acid aspartic acid, N,N′-diacetic acid alanine, N,N′-diacetic acid aspartic acid, N-monoacetic acid, iminodisuccinic acid, diaminoalkyldi(sulfosuccinic) acids (DDS), ethylenediamine-NN′-bis(ortho-hydroxyphenyl-acetic acid)), N,N′-bis(2-hydroxybenzyl)ethylenediamine-N,N′-diacetic acid, ethylenediamine tetraproprionate, triethylenetetraamine hexaacetate, diethylenetriamine pentaacetate, dipicolinic acid, ethylenedicysteic acid (EDC), ethylenediamine-N,N′-bis(2-hydroxyphenylacetic acid) (EDDHA), glutamic diacetic acid (GLDA), hexadentateaminocarboxylate (HBED), polyethyleneimine, 1-hydroxydiphosphonate, aminotri(methylenephosphonic acid) (ATMP), nitrilotrimethylene phosphonate (NTP), ethylenediamine tetramethylene phosphonate (EDTMP), diethylene triamine pentamethylene phosphonate (DTPMP), 1-hydroxy-ethane-1,1-diphosphonate (HEDP), 2-phosphonobutane-1,2,4-tricarboxylic acid, polyphosphoric acid, sodium tripolyphosphate, tetrasodium diphosphate, hexametaphosphoric acid, sodium metaphosphate, phosphonic acid, 1,2-dihydroxy-3,5-disulfobenzene.

In one embodiment, the composition for treating keratin fibers according to the invention comprises at least one chelating agent chosen from the group consisting of N-hydroxysuccinimide, EDTA salts, nitrilotriacetic acid (NTA), deferoxamine, hydroxamic acids and their salts, phytic acid, phytate, gluconic acid and its salts, transferrin, lactoferrin, furildioxime and their derivatives.

In one embodiment, the composition for treating keratin fibers according to the invention comprises at least one chelating agent chosen from the group consisting of EDTA salts, nitrilotriacetic acid (NTA), 1-hydroxy-ethane-1,1-diphosphonate (HEDP), salts of ethylenediaminetetramethylenephosphonate (EDTMP), dipicolinic acid, salts of diethylenetriaminepentamethylenephosphonate (DTPMP).

In one embodiment, the composition for treating keratin fibers according to the invention is characterized in that the mass proportion of the at least one chelating agent is between 0.00001 and 10% relative to the total mass of the composition.

In one embodiment, the composition for treating keratin fibers according to the invention is characterized in that the mass proportion of the at least one chelating agent is between 0.001 and 5% relative to the total mass of the composition.

In one embodiment, the composition for treating keratin fibers according to the invention comprises at least one propellant.

In one embodiment, the composition for treating keratin fibers according to the invention comprises at least one propellant chosen from the group consisting of compounds with a boiling point between −45° C. and 5° C.

In one embodiment, the composition for treating keratin fibers according to the invention comprises at least one propellant chosen from the group consisting of compounds having a vapor pressure of between 1.17 Bar and 7.45 Bar at 21.1° C.

In one embodiment, the composition for treating keratin fibers according to the invention comprises at least one propellant chosen from the group consisting of compounds having a vapor pressure of between 1.17 Bar and 4.83 Bar at 21.1° C.

In one embodiment, the composition for treating keratin fibers according to the invention comprises at least one propellant chosen from the group consisting of compounds having a vapor pressure of between 2.14 Bar and 3.79 Bar at 21.1° C.

In one embodiment, the propellant may be in liquefied form in the pressurized aerosol container.

In one embodiment, the propellant may be in compressed form in the pressurized aerosol container.

In one embodiment, the composition for treating keratin fibers according to the invention comprises at least one propellant chosen from the group consisting of chemically inert hydrocarbons, halogenated hydrocarbons, air, nitrogen, oxygen and nitrous oxide.

In one embodiment, the composition for treating keratin fibers according to the invention comprises at least one chemically inert hydrocarbon propellant chosen from the group consisting of methane, propane, propene, n-butane, isobutane, isobutene, n-pentane, pentene, isopentane, ethane and cyclopropane.

In one embodiment, the composition for treating keratin fibers according to the invention comprises at least one propellant of halogenated hydrocarbon type chosen from the group consisting of dichlorodifluoromethane, 1,1-dichloro-1,1,2,2-tetrafluoroethane, 1-chloro-1,1-difluoro-2,2-trifluoroethane, 1-chloro-1,1-difluoroethylene, 1,1-difluoroethane, dimethyl ether, monochlorodifluoromethane, trans-1,3,3,3-tetrafluoropropene, 1,1,1,3-tetrafluoroethane, heptafluoro-n-propane and perfluoromethane,.

In one embodiment, the propellant is dimethyl ether.

In one embodiment, the propellant is n-butane.

In one embodiment, the composition for treating keratin fibers according to the invention is characterized in that the mass proportion of the at least one propellant is between 1 and 10% relative to the total mass of the composition.

In one embodiment, the composition for treating keratin fibers according to the invention is characterized in that the mass proportion of the at least one propellant is between 2 and 8% relative to the total mass of the composition.

In one embodiment, the composition for treating keratin fibers according to the invention comprises at least one anti-dandruff agent.

In one embodiment, the composition for treating keratin fibers according to the invention comprises at least one anti-dandruff agent chosen from the group consisting of antimicrobials, antibacterials and antifungals.

In one embodiment, the composition for treating keratin fibers according to the invention comprises at least one anti-dandruff agent chosen from the group consisting of antimicrobials.

In one embodiment, the composition for treating keratin fibers according to the invention comprises at least one anti-dandruff agent chosen from the group consisting of antibacterials.

In one embodiment, the composition for treating keratin fibers according to the invention comprises at least one anti-dandruff agent chosen from the group consisting of antifungals.

In one embodiment, the composition for treating keratin fibers according to the invention comprises at least one anti-dandruff agent selected from the group consisting of zinc pyrithione, camphor, sulfur, selenium sulfide, coal tar, Castellani solution, aluminum chloride, gentian violet, triclosan, triclocarban, undecylenic acid, and its metal salts, potassium permanganate, selenium sulfide, sodium thiosulfate, propylene glycol, bitter orange oil, tea tree oil, clove leaf essential oil, coriander, red thyme, cinnamon essence, cinnamic aldehyde, citronellic acid, hinokitol, pale ichthyol, azelaic acid, lyticase, iodopropynyl butylcarbamate (IPBC), octyl isothiazolinone and charcoal.

In one embodiment, the composition for treating keratin fibers according to the invention comprises at least one anti-dandruff agent chosen from the group consisting of polyvalent metal salts of pyrithione.

In one embodiment, the composition for treating keratin fibers according to the invention comprises at least one anti-dandruff agent chosen from the group consisting of azoles, keratolytic agents or desquamation actives, hydroxypyridones, strobilurins and metal chelators.

In one embodiment, the composition for treating keratin fibers according to the invention comprises at least one azole anti-dandruff agent chosen from the group consisting of climbazole, ketoconazole, itraconazole, econazole and elubiol.

In one embodiment, the composition for treating keratin fibers according to the invention comprises at least one azole anti-dandruff agent chosen from the group consisting of imidazoles and triazoles.

In one embodiment, the composition for treating keratin fibers according to the invention comprises at least one imidazole-type azole anti-dandruff agent selected from the group consisting of benzimidazole, benzothiazole, bifonazole, butaconazole nitrate, climbazole, clotrimazole, croconazole, eberconazole, econazole, elubiol, fenticonazole nitrate, fluconazole, flutimazole, isoconazole, ketoconazole, lanoconazole, metronidazole, miconazole, neticonazole, omoconazole, oxiconazole nitrate, sertaconazole, sulconazole nitrate and tioconazole.

In one embodiment, the composition for treating keratin fibers according to the invention comprises at least one triazole-type anti-dandruff agent chosen from the group consisting of terconazole and itraconazole.

In one embodiment, the composition for treating keratin fibers according to the invention comprises at least one hydroxypyridone anti-dandruff agent chosen from the group consisting of piroctone olamine, ciclopirox olamine, rilopirox and MEA-hydroxyoctyloxypyridinone.

In one embodiment, the composition for treating keratin fibers according to the invention comprises at least one anti-dandruff agent keratolytic agent chosen from the group consisting of salicylic acid and alpha hydroxy acids.

In one embodiment, the composition for treating keratin fibers according to the invention comprises at least one keratolytic agent selected from the group consisting of 2-hydroxyethanoic acid (glycolic acid), 2-hydroxypropanoic acid (lactic acid), 2-methyl-2-hydroxypropanoic acid (methyllactic acid), 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid (alphahydroxylauric acid), 2-hydroxytetradecanoic acid (alpha-hydroxymyristic acid), 2-hydroxyhexadecanoic acid (alpha-hydroxypalmitic acid), 2-hydroxyoctadecanoic acid (alpha-hydroxystearic acid), 2-hydroxyeicosanoic acid (alpha-hydroxyarachidonic acid), 2-phenyl-2-hydroxyethanoic acid (mandelic acid), 2,2-diphenyl-2-hydroxyethanoic acid (benzylic acid), 3-phenyl-2-hydroxypropanoic acid (phenyllactic acid), 2-phenyl-2-methyl-2-hydroxyethanoic acid (atrolactic acid), 2-(4′-hydroxyphenyl) 2-hydroxyethanoic acid, 2-(4′-chlorophenyl-2-hydroxyethanoic acid, 2-(3′-hydroxy-4′-methoxyphenyl)-2-hydroxyethanoic acid, 2-(4′-hydroxy-3′-methoxyphenyl)-2-hydroxyethanoic acid, 3′-(2-hydroxyphenyl)-2-hydroxypropanoic acid, 3-(4′-hydroxyphenyl)-2-hydroxypropanoic acid acid, 2-(3′,4′-dihydroxyphenyl), 2-hydroxyethanoic acid, 5-n-octanoylsalicylic acid, 5-n-dodecanoylsalicylic acid, 5-n-decanoylsalicylic acid, 5-n-octylsalicylic acid, 5-n-heptyloxysalicylic acid, 4-n-heptyloxysalicylic acid, 2-hydroxy-3-methylbenzoic acid and 2-hydroxy-3-methyoxybenzoic acid.

In one embodiment, the composition for treating keratin fibers according to the invention comprises at least one keratolytic agent selected from the group consisting of glycolic acid, tartaric acid, salicylic acid, citric acid, lactic acid, pyruvic acid, gluconic acid, glucuronic acid, malic acid, mandelic acid, oxalic acid, malonic acid, succinic acid, acetic acid, phenol, resorcinol, retinoic acid, adapalene, trichloroacetic acid, 5-fluorouracil, and azelaic acid.

In one embodiment, the composition for treating keratin fibers according to the invention is characterized in that the mass proportion of the at least one anti-dandruff agent is between 0.001 and 10% relative to the total mass of the composition.

In one embodiment, the composition for treating keratin fibers according to the invention is characterized in that the mass proportion of the at least one anti-dandruff agent is between 0.01 and 5% relative to the total mass of the composition.

In one embodiment, the composition for treating keratin fibers according to the invention is characterized in that the mass proportion of the at least one anti-dandruff agent is between 0.05 and 2% relative to the total mass of the composition.

In one embodiment, the composition for treating keratin fibers according to the invention comprises at least one physiologically acceptable medium, that is to say a medium compatible with human keratin materials such as the skin (of the body, face, eye contour or scalp), hair, eyelashes, eyebrows, body hair, nails or lips.

In one embodiment, the physiologically acceptable medium is selected from the group consisting of water or a mixture of water and at least one cosmetically acceptable solvent.

In one embodiment, the composition for treating keratin fibers according to the invention comprises at least one cosmetically acceptable solvent.

In one embodiment, the composition for treating keratin fibers according to the invention comprises at least one cosmetically acceptable solvent chosen from the group of lower C₂-C₄ alcohols.

In one embodiment, the C₂-C₄ lower alcohol is selected from the group consisting of ethanol and isopropanol.

In one embodiment, the composition for treating keratin fibers according to the invention comprises at least one cosmetically acceptable solvent chosen from the group of polyols.

In one embodiment, the composition for treating keratin fibers according to the invention comprises at least one cosmetically acceptable solvent chosen from the group of C₂-C₆ polyols.

In one embodiment, the C₂-C₆ polyol is selected from the group consisting of glycerol or glycerin, propylene glycol, butylene glycol, pentylene glycol, hexylene glycol, dipropylene glycol and diethylene glycol.

In one embodiment, the composition for treating keratin fibers according to the invention comprises at least one cosmetically acceptable solvent chosen from the group of polyol ethers.

In one embodiment, the polyol ether is selected from the group consisting of 2-butoxyethanol, propylene glycol monomethyl ether and diethylene glycol monomethyl or monoethyl ether, alone or in a mixture.

In one embodiment, the composition for treating keratin fibers according to the invention comprises a solvent chosen from the group consisting of water, C₂-C₄ alcohols, polyols and polyol ethers, alone or as a mixture.

The compositions according to the invention are intended to be used in formulations traditionally used as hair treatment agents.

The formulations based on compositions according to the invention can be in the form of a solution, dispersion, oil/water emulsion, water/oil emulsion, water/oil/water emulsion, powder, talc, capsules, sponge, foam, lacquer and gel.

The compositions of the invention may be used in hair toners, hair dyes, hair styling products, shampoos, hair conditioners, hair conditioners, conditioning shampoos, hair treatment products, hair sprays, hair masks, hair care products, hair treatment products, perm fixing solutions, hair shaping products, shampoos for colored hair, hair fixatives, hair holding products, hair styling preparations, leave-in hair products, curling iron lotions, setting mousses, hair gels, hair waxes or combinations thereof.

### EXAMPLES

The following examples are given to illustrate the invention.

Process for the preparation of hair serum formulations according to the invention

Phase 1 ingredients are dispersed in Ultra-Turrax™.

Phases 1, 2 and 3 are then mixed to obtain a hair serum.

### Example 1: Composition of a Hair Serum According to the Invention

TABLE 1

| | | | % by |
|---|---|---|---|
| Phase | Trade name | INCI | mass |
| 1 | H110 from BRB | DIMETHICONOL | 8.8 |
| | CYCLODODECANE | | 8 |
| 2 | 2,3,6-TRIMETHYLHEPTANE | | 38 |
| | N-DODECANE | DODECANE | 45 |
| 3 | KARILEINE OIL | BUTYROSPERMUM PARKII | 0.2 |
| | BIOSYNTHIS | (SHEA) BUTTER | |

| | |
|---|---|
| TOTAL | 100 |

### Example 2: Composition of a Hair Serum According to the Invention

TABLE 2

| | | | % by |
|---|---|---|---|
| Phase | Trade name | INCI | mass |
| 1 | H110 from BRB | DIMETHICONOL | 7.2 |
| | VEGETABLE | OLEA EUROPAEA (OLIVE) | 7.5 |
| | SQUALENE ST | OIL UNSAPONIFIABLES | |
| | BIOSYNTHIS | | |
| | CYCLODODECANE | | 7.5 |
| 2 | N-DODECANE | DODECANE | 37 |
| | 2,3,6-TRIMETHYLHEPTANE | | 38 |
| 3 | KARILEINE OIL | BUTYROSPERMUM PARKII | 2.8 |
| | BIOSYNTHIS | (SHEA) BUTTER | |

| | |
|---|---|
| TOTAL | 100 |

### Example 3: Composition of a Hair Serum According to the Invention

TABLE 3

| | | | % by |
|---|---|---|---|
| Phase | Trade name | INCI | mass |
| 1 | BELSIL DL 1,000,000 | DIMETHICONE | 12 |
| | H110 of BRB | DIMETHICONOL | 5 |
| 2 | N-DODECANE | DODECANE | 40 |
| | 2,3,6-TRIMETHYLHEPTANE | | 40 |
| 3 | KARILEINE OIL | BUTYROSPERMUM PARKII | 3 |
| | BIOSYNTHIS | (SHEA) BUTTER | |

| | |
|---|---|
| TOTAL | 100 |

### Example 4: Composition of a Hair Serum According to the Invention

TABLE 4

| | | | % by |
|---|---|---|---|
| Phase | Trade name | INCI | mass |
| 1 | BELSIL DL 1,000,000 | DIMETHICONE | 12 |
| | H110 from BRB | DIMETHICONOL | 5 |
| | VEGETABLE | OLEA EUROPAEA (OLIVE) | 6 |
| | SQUALENE ST | OIL UNSAPONIFIABLES | |
| | BIOSYNTHIS | | |
| | VEGELIGHT 1214 | COCONUT ALKANE | 19 |
| 2 | 2,3,6-TRIMETHYLHEPTANE | | 25 |
| | N-DODECANE | DODECANE | 30 |
| 3 | KARILEINE OIL | BUTYROSPERMUM PARKII | 3 |
| | BIOSYNTHIS | (SHEA) BUTTER | |

| | |
|---|---|
| TOTAL | 100 |

### Example 5: Influence of a Branched Alkane on the Viscosity of a Composition According to the Invention

Compositions comprising 20% dimethiconol, 70% fraction A and 30% fraction B.
The nature of the constituents and the results of the kinematic viscosity and IR measurements are given in Table 5, below.

TABLE 5

| | | | VISCOSITY | IR |
|---|---|---|---|---|
| DIMETHICONOL | FRACTION A | FRACTION B | (25° C.) | (25° C.) |
| DOWSIL 1515 | 2,3,6- | N-DODECANE | 5310 cST | 1.4118 |
| | TRIMETHYLHEPTANE | | | |
| DOWSIL 1515 | N-DECANE | N-DODECANE | 3977 cST | 1.4115 |

The viscosities obtained are higher than 1300 cSt which is unpredictable and extremely interesting in a composition intended for hair because viscosity is a fundamental criterion.

The IR refractive indices are comparable.

### Example 6: Sensory Evaluation

A composition according to the prior art was prepared. It corresponds to the formulation according to Example 1, but the trimethyl heptane was replaced by N-DECANE.

TABLE 6

| | | | % by |
|---|---|---|---|
| Phase | Trade name | INCI | mass |
| 1 | H110 from BRB | DIMETHICONOL | 8.8 |
| | CYCLODODECANE | | 8 |
| 2 | N DECANE | | 38 |
| | N-DODECANE | DODECANE | 45 |
| 3 | KARILEINE OIL | BUTYROSPERMUM PARKII | 0.2 |
| | BIOSYNTHIS | (SHEA) BUTTER | |

| | |
|---|---|
| TOTAL | 100 |

The compositions according to example 1 and according to the prior art described above were applied to locks of sensitized Caucasian hair, at a rate of 0.1 g of composition per gram of hair.

A sensory evaluation of the shine is then carried out by 3 experts for each of the strands.

The locks of hair treated with the composition according to the invention have a better level of shine than the locks treated with the composition according to the prior art.

They also have a smoother and less greasy feel than the strands treated with the composition according to the prior art.

1-6. (canceled)

7. A composition for the cosmetic treatment of keratin fibers wherein it comprises:
(a) a fraction A which comprises at least one C₁₀ branched alkane having a flash point less than or equal to 95° C. measured according to standard ASTM D93,
(b) a fraction B which comprises at least one volatile linear alkane chosen from a group comprising C₇, C₈, C₉, C₁₀, C₁₁, C₁₂, C₁₃ alkanes and their mixtures or branched chosen from a group comprising branched C₁₂, branched C₁₃, branched C₁₄, branched C₁₅, branched C₁₆ alkanes and their mixtures, the fraction B having a flash point less than or equal to 95° C. measured according to standard ASTM D93, in C>10,
(c) at least one dimethiconol
and wherein it is free from volatile silicone oil and isododecane.

8. The composition according to claim 7, wherein the flash point measured according to standard ASTM D93 of the mixture consisting of fraction A and fraction B is less than or equal to 50° C.

9. The composition according to claim 7, wherein the mass percentage of fraction A is between 20 and 100% relative to the mass of the mixture consisting of fraction A and fraction B.

10. The composition according to claim 7, wherein the at least one branched alkane of fraction A is a trimethylheptane.

11. The composition according to claim 10, wherein the trimethylheptane is 2,3,6-trimethylheptane (CAS 4032-93-3).

12. A method comprising treating keratin fibers with a composition according to claim 7.
